SEQUENCE LISTING (12 sequences)

SEQ ID NO: 1 - RNA, 1063 nt, Mus musculus
  source 1..1063: mol_type = mRNA
agagcagaccccgcccgcgcaaggaaaggagggaggtccaccggtatccactatgcttctggaagaagtctgcgtcggcgaccggttgagtggcgcagcggcccgtggcgacgtgcaagaggtccgccgccttcttcaccgggagctggtgcatcctgacgccctgaaccgctttggcaagacggccttgcaggtcatgatgtttggaagtccagcagttgctttggagctcctgaagcaaggtgccagccccaatgtccaagatgcctccggtactagtcctgtgcatgatgcggctcgcaccgggttcctggacaccctgaaggttctggtggagcatggtgctgatgtcaatgccctggacagcactgggtcgctccccatccatctggcgataagagagggccatagctccgtggtcagcttcctagctcctgaatctgatctccaccacagggacgcttccggtctcactcccctggagttggctcggcagagaggggctcagaacctcatggacattctgcaggggcacatgatgatcccaatgtgacccaaggccactgtctccagccttactgggttacttgtcaacaaaagaggaaagaaactttctcttttcacacctgtccattgaagaagggagtgggaggagcagtttgtggtttattggtgttgatttcttgagtgtgtgtgtttggggggtgtttctcatttgtttttctcaccccttttggtgtgttggacaaagaaggggctcctacaggcaacagccatctaaacggttcagtttcctctgcactggggctgcaccagggcaggggtttaaagccctagcctcagagtgaggtcatcacttcccgggccccttggaagctggtgaccttggcaggctgtgctcagagagccctgaagtgtgagccatctactttgggcatgtgagggggaagggaaagcatttcaaatcaattaaaaggataacatgagttcatttttctttcttggaaggtttccagtctgttgtacagagtttgaaatataaatatttattgctttatggaaa

SEQ ID NO: 2 - protein, 166 aa, Mus musculus
MLLEEVCVGDRLSGAAARGDVQEVRRLLHRELVHPDALNRFGKTALQVMMFGSPAVALELLKQGASPNVQDASGTSPVHDAARTGFLDTLKVLVEHGADVNALDSTGSLPIHLAIREGHSSVVSFLAPESDLHHRDASGLTPLELARQRGAQNLMDILQGHMMIPM

SEQ ID NO: 3 - RNA, 1115 nt, Mus musculus
  source 1..1115: mol_type = mRNA
agagcagaccccgcccgcgcaaggaaaggagggaggtccaccggtatccactatgcttctggaagaagtctgcgtcggcgaccggttgagtggcgcagcggcccgtggcgacgtgcaagaggtccgccgccttcttcaccgggagctggtgcatcctgacgccctgaaccgctttggcaagacggccttgcaggtgccctcttgaaggatctctggttatttcagggacatcttcatttcggcaggtcatgatgtttggaagtccagcagttgctttggagctcctgaagcaaggtgccagccccaatgtccaagatgcctccggtactagtcctgtgcatgatgcggctcgcaccgggttcctggacaccctgaaggttctggtggagcatggtgctgatgtcaatgccctggacagcactgggtcgctccccatccatctggcgataagagagggccatagctccgtggtcagcttcctagctcctgaatctgatctccaccacagggacgcttccggtctcactcccctggagttggctcggcagagaggggctcagaacctcatggacattctgcaggggcacatgatgatcccaatgtgacccaaggccactgtctccagccttactgggttacttgtcaacaaaagaggaaagaaactttctcttttcacacctgtccattgaagaagggagtgggaggagcagtttgtggtttattggtgttgatttcttgagtgtgtgtgtttggggggtgtttctcatttgtttttctcaccccttttggtgtgttggacaaagaaggggctcctacaggcaacagccatctaaacggttcagtttcctctgcactggggctgcaccagggcaggggtttaaagccctagcctcagagtgaggtcatcacttcccgggccccttggaagctggtgaccttggcaggctgtgctcagagagccctgaagtgtgagccatctactttgggcatgtgagggggaagggaaagcatttcaaatcaattaaaaggataacatgagttcatttttctttcttggaaggtttccagtctgttgtacagagtttgaaatataaatatttattgctttatggaaa

SEQ ID NO: 4 - protein, 118 aa, Mus musculus
MMFGSPAVALELLKQGASPNVQDASGTSPVHDAARTGFLDTLKVLVEHGADVNALDSTGSLPIHLAIREGHSSVVSFLAPESDLHHRDASGLTPLELARQRGAQNLMDILQGHMMIPM

SEQ ID NO: 5 - RNA, 1245 nt, Mus musculus
  source 1..1245: mol_type = mRNA
agctgcgagcgctggggtcgtacgggcggagggcgcaggctctgaggccggcaaatgatcatagagttggccctggtggcaccgcagtccctagagttctgatccagctcttgctggttccccagccctgaccttaactgggcttggggctgggtgggtttcacagtccaccggtatccactatgcttctggaagaagtctgcgtcggcgaccggttgagtggcgcagcggcccgtggcgacgtgcaagaggtccgccgccttcttcaccgggagctggtgcatcctgacgccctgaaccgctttggcaagacggccttgcaggtgccctcttgaaggatctctggttatttcagggacatcttcatttcggcaggtcatgatgtttggaagtccagcagttgctttggagctcctgaagcaaggtgccagccccaatgtccaagatgcctccggtactagtcctgtgcatgatgcggctcgcaccgggttcctggacaccctgaaggttctggtggagcatggtgctgatgtcaatgccctggacagcactgggtcgctccccatccatctggcgataagagagggccatagctccgtggtcagcttcctagctcctgaatctgatctccaccacagggacgcttccggtctcactcccctggagttggctcggcagagaggggctcagaacctcatggacattctgcaggggcacatgatgatcccaatgtgacccaaggccactgtctccagccttactgggttacttgtcaacaaaagaggaaagaaactttctcttttcacacctgtccattgaagaagggagtgggaggagcagtttgtggtttattggtgttgatttcttgagtgtgtgtgtttggggggtgtttctcatttgtttttctcaccccttttggtgtgttggacaaagaaggggctcctacaggcaacagccatctaaacggttcagtttcctctgcactggggctgcaccagggcaggggtttaaagccctagcctcagagtgaggtcatcacttcccgggccccttggaagctggtgaccttggcaggctgtgctcagagagccctgaagtgtgagccatctactttgggcatgtgagggggaagggaaagcatttcaaatcaattaaaaggataacatgagttcatttttctttcttggaaggtttccagtctgttgtacagagtttgaaatataaatatttattgctttatggaaa

SEQ ID NO: 6 - protein, 118 aa, Mus musculus; identical to SEQ ID NO: 4

SEQ ID NO: 7 - RNA, 1425 nt, Homo sapiens
  source 1..1425: mol_type = mRNA
agagcagaccccgcccggcgaggaggagggagggtgagttagggggagacccggcccccaaggggcgggcgccgggcagggccccgcgggcggccgagggttgggcccggctcccagcccctcgccgtcctccggctgacagggggaggagcccgccgggagggccggggtctcgggctggggagccgggacgggagagcagcgcagccgggtgcaccgcggccgcgccccgggagggctgttcgggccagcgcccgccggctgctccgcgctgacagcgccgggctggggcggggcggggggctttgcaggccgccagtgtcgacatgctgctggaggaggttcgcgccggcgaccggctgagtggggcggcggcccggggcgacgtgcaggaggtgcgccgccttctgcaccgggagctggtgcatcccgacgccctcaaccgcttcggcaagacggcgctgcaggtcatgatgtttggcagcaccgccatcgccctggagctgctgaagcaaggtgccagccccaatgtccaggacacctccggtaccagtccagtccatgacgcagcccgcactggattcctggacaccctgaaggtcctagtggagcacggggctgatgtcaacgtgcctgatggcaccggggcacttccaatccatctggcagttcaagagggtcacactgctgtggtcagctttctggcagctgaatctgatctccatcgcagggacgccaggggtctcacacccttggagctggcactgcagagaggggctcaggacctcgtggacatcctgcagggccacatggtggccccgctgtgatctggggtcaccctctccagcaagagaaccccgtggggttatgtatcagaagagaggggaagaaacactttctcttcttgtttctcctgcccactgctgcagtaggggaggagcacagtttgtggcttataggtgttggttttgggggtgtgagtgtttgggggacgtttctcatttgtttttctcactccttttggtgtgttggacagagaagggctcctgcaggccacagccacctaaacggttcagtttcttctgcgcctcaggctgctggggcctcagacgagacccaagggcagagcatttaagagtgaagtcatgacctccagggagcctagaagctggtggccttggccggctgtgctcagagacctgaagtgtgcacgttgcttcaggcatggggggtggggggagcgtcccaaatcaataagaaggtagaatgagttatgagttattcatattctgttggaagcttgttttccagtctcttgtacagcgttttaaaagaaatggattctatttattatgctttattggaaaaaatgttgtaataatttaatgtttttacccattaaattaagacttgtgcatgatcaca

SEQ ID NO: 8 - RNA, 1147 nt, Homo sapiens
  source 1..1147: mol_type = mRNA
agagcagaccccgcccggcgaggaggagggagggccgccagtgtcgacatgctgctggaggaggttcgcgccggcgaccggctgagtggggcggcggcccggggcgacgtgcaggaggtgcgccgccttctgcaccgggagctggtgcatcccgacgccctcaaccgcttcggcaagacggcgctgcaggtcatgatgtttggcagcaccgccatcgccctggagctgctgaagcaaggtgccagccccaatgtccaggacacctccggtaccagtccagtccatgacgcagcccgcactggattcctggacaccctgaaggtcctagtggagcacggggctgatgtcaacgtgcctgatggcaccggggcacttccaatccatctggcagttcaagagggtcacactgctgtggtcagctttctggcagctgaatctgatctccatcgcagggacgccaggggtctcacacccttggagctggcactgcagagaggggctcaggacctcgtggacatcctgcagggccacatggtggccccgctgtgatctggggtcaccctctccagcaagagaaccccgtggggttatgtatcagaagagaggggaagaaacactttctcttcttgtttctcctgcccactgctgcagtaggggaggagcacagtttgtggcttataggtgttggttttgggggtgtgagtgtttgggggacgtttctcatttgtttttctcactccttttggtgtgttggacagagaagggctcctgcaggccacagccacctaaacggttcagtttcttctgcgcctcaggctgctggggcctcagacgagacccaagggcagagcatttaagagtgaagtcatgacctccagggagcctagaagctggtggccttggccggctgtgctcagagacctgaagtgtgcacgttgcttcaggcatggggggtggggggagcgtcccaaatcaataagaaggtagaatgagttatgagttattcatattctgttggaagcttgttttccagtctcttgtacagcgttttaaaagaaatggattctatttattatgctttattggaaaaaatgttgtaataatttaatgtttttacccattaaattaagacttgtgcatgatcaca

SEQ ID NO: 9 - protein, 166 aa, Homo sapiens
MLLEEVRAGDRLSGAAARGDVQEVRRLLHRELVHPDALNRFGKTALQVMMFGSTAIALELLKQGASPNVQDTSGTSPVHDAARTGFLDTLKVLVEHGADVNVPDGTGALPIHLAVQEGHTAVVSFLAAESDLHRRDARGLTPLELALQRGAQDLVDILQGHMVAPL

SEQ ID NO: 10 - RNA, 1193 nt, Mus musculus
  source 1..1193: mol_type = mRNA
agctgcgagcgctggggtcgtacgggcggagggcgcaggctctgaggccggcaaatgatcatagagttggccctggtggcaccgcagtccctagagttctgatccagctcttgctggttccccagccctgaccttaactgggcttggggctgggtgggtttcacagtccaccggtatccactatgcttctggaagaagtctgcgtcggcgaccggttgagtggcgcagcggcccgtggcgacgtgcaagaggtccgccgccttcttcaccgggagctggtgcatcctgacgccctgaaccgctttggcaagacggccttgcaggtcatgatgtttggaagtccagcagttgctttggagctcctgaagcaaggtgccagccccaatgtccaagatgcctccggtactagtcctgtgcatgatgcggctcgcaccgggttcctggacaccctgaaggttctggtggagcatggtgctgatgtcaatgccctggacagcactgggtcgctccccatccatctggcgataagagagggccatagctccgtggtcagcttcctagctcctgaatctgatctccaccacagggacgcttccggtctcactcccctggagttggctcggcagagaggggctcagaacctcatggacattctgcaggggcacatgatgatcccaatgtgacccaaggccactgtctccagccttactgggttacttgtcaacaaaagaggaaagaaactttctcttttcacacctgtccattgaagaagggagtgggaggagcagtttgtggtttattggtgttgatttcttgagtgtgtgtgtttggggggtgtttctcatttgtttttctcaccccttttggtgtgttggacaaagaaggggctcctacaggcaacagccatctaaacggttcagtttcctctgcactggggctgcaccagggcaggggtttaaagccctagcctcagagtgaggtcatcacttcccgggccccttggaagctggtgaccttggcaggctgtgctcagagagccctgaagtgtgagccatctactttgggcatgtgagggggaagggaaagcatttcaaatcaattaaaaggataacatgagttcatttttctttcttggaaggtttccagtctgttgtacagagtttgaaatataaatatttattgctttatggaaa

SEQ ID NO: 11 - DNA, 2517 bp, Homo sapiens
  source 1..2517: mol_type = genomic DNA
agagcagaccccgcccggcgaggaggagggagggtgagttagggggagacccggcccccaaggggcgggcgccgggcagggccccgcgggcggccgagggttgggcccggctcccagcccctcgccgtcctccggctgacagggggaggagcccgccgggagggccggggtctcgggctggggagccgggacgggagagcagcgcagccgggtgcaccgcggccgcgccccgggagggctgttcgggccagcgcccgccggctgctccgcgctgacagcgccgggctggggcggggcggggggctttgcaggccgccagtgtcgacatgctgctggaggaggttcgcgccggcgaccggctgagtggggcggcggcccggggcgacgtgcaggaggtgcgccgccttctgcaccgcgagctggtgcatcccgacgccctcaaccgcttcggcaagacggcgctgcaggtgaggccgggccggtccaggttggggcggggcggctaagcgggatgaggatcgagaccccagagctcctctctggtctcctggccctggtggtgacccactgggaaccggtccttctctaggctttcattgttcccccacagtcccccttcgaaggtgggtggtgggtgaggctaggttgtggggggaggggagagctttttagcatcttctctgggaggattcctggttctctgagtgccggggattcactttccctggggaggttcttattccgtgggtgcgggttcagctcctttggatgccgtttccatggggacggctcacgtttcttggggcttctgtggggagatccgatctctggaaggagaggtcctgtctctaggggttcctcattttgtagggggctctctgagacaaatctggttacttgggggtggggtcttcattcctaaaggttatgcggatccttgatatagtatgggggccccaatggaggaacaatgtcagattgcaagacatccctatattccctgggagacattttacaggggagtctcgtttctcttttattattacttttggaatcccgcttcttgaaaggagctctcaggtactctgaggtgacatcaccattatgtaggaggaggctatttcctggagaaggggttcgctttcctttgacatcttttttcccttatgaggactatcaattctttgggggtagttctccatctctgggggcccatttcctggaggggattttctgtttcctggtgattgcccatatctgggggcgtctttggtcccttgggggagagtcttaggttccttgaaagtttccatggggggctgttctctgggttggggggtctcattcttaagggagtgaccagcttttggggggggtccctattcccagaagaactttcctgggggcttatgcagcttctccgttcactagaggacccctcctctagcatttcctgtttctgggagatgcctctttctggatgacccttctctttccagtgaccccttttgtggaacctcctcccttgaggacctcctgatcctctgtccctcacacaggtcatgatgtttggcagcaccgccatcgccctggagctgctgaagcaaggtgccagccccaatgtccaggacacctccggtaccagtccagtccatgacgcagcccgcactggattcctggacaccctgaaggtcctagtggagcacggggctgatgtcaacgtgcctgatggcaccggggcacttccaatccatctggcagttcaagagggtcacactgctgtggtcagctttctggcagctgaatctgatctccatcgcagggacgccaggggtctcacacccttggagctggcactgcagagaggggctcaggacctcgtggacatcctgcagggccacatggtggccccgctgtgatctggggtcaccctctccagcaagagaaccccgtggggttatgtatcagaagagaggggaagaaacactttctcttcttgtttctcctgcccactgctgcagtaggggaggagcacagtttgtggcttataggtgttggttttgggggtgtgagtgtttgggggacgtttctcatttgtttttctcactccttttggtgtgttggacagagaagggctcctgcaggccacagccacctaaacggttcagtttcttctgcgcctcaggctgctggggcctcagacgagacccaagggcagagcatttaagagtgaagtcatgacctccagggagcctagaagctggtggccttggccggctgtgctcagagacctgaagtgtgcacgttgcttcaggcatggggggtggggggagcgtcccaaatcaataagaaggtagaatgagttatgagttattcatattctgttggaagcttgttttccagtctcttgtacagcgttttaaaagaaatggattctatttattatgctttattggaaaaaatgttgtaataatttaatgtttttacccattaaattaagacttgtgcatgatcaca

SEQ ID NO: 12 - DNA, 2520 bp, Homo sapiens
  source 1..2520: mol_type = genomic DNA
agagcagaccccgcccggcgaggaggagggagggtgagttagggggagacccggcccccaaggggcgggcgccgggcagggccccgcgggcggccgagggttgggcccggctcccagcccctcgccgtcctccggctgacagggggaggagcccgccgggagggccggggtctcgggctggggagccgggacgggagagcagcgcagccgggtgcaccgcggccgcgccccgggagggctgttcgggccagcgcccgccggctgctccgcgctgacagcgccgggctggggcggggcggggggctttgcaggccgccagtgtcgacatgctgctggaggaggttcgcgccggcgaccggctgagtggggcggcggcccggggcgacgtgcaggaggtgcgccgccttctgcaccgggagctggtgcatcccgacgccctcaaccgcttcggcaagacggcgctgcaggtgaggccgggccggtccaggttggggcggggcggctaagcgggatgaggatcgagaccccagagctcctctctggtctcctggccctggtggtgacccactgggaaccggtccttcttctctaggctttcattgttcccccacagtcccccttcgaaggtgggtggtgggtgaggctaggttgtggggggaggggagagctttttagcatcttctctgggaggattcctggttctctgagtgccggggattcactttccctggggaggttcttattccgtgggtgcgggttcagctcctttggatgccgtttccatggggacggctcacgtttcttggggcttctatggggagatccgatctctggaaggagaggtcctgtctctaggggttcctcattttgtagggggctctctgagacaaatctggttacttgggggtggggtcttcattcctaaaggttatgcggatccttgatatagtatgggggccccaatggaggaacaatgtcagattgcaagacatccctatattccctgggagacattttacagggaagtctcgtttctcttttattattacttttggaatcccgcttcttgaaaggagctctcaggtactctgaggtgacatcaccattatgtaggaggaggctatttcctggagaaggggttcgctttcctttgacatcttttttcccttatgaggactatcaattctttgggggtagttctccatctctgggggcccatttcctggaggggattttctgtttcctggtgattgcccatatctgggggcgtctttggtcccttgggggagagtcttaggttccttgaaagtttccatggggggctgttctctgggttggggggtctcattcttaagggagtgaccagcttttggggggggtccctattcccagaagaactttcctgggggcttatgcagcttctccgttcactagaggacccctcctctagcatttcctgtttctgggagatgcctctttctggatgacccttctctttccagtgaccccttttgtggaacctcctcccttgaggacctcctgatcctctgtccctcacacaggtcatgatgtttggcagcaccgccatcgccctggagctgctgaagcaaggtgccagccccaatgtccaggacacctccggtaccagtccagtccatgacgcagcccgcactggattcctggacaccctgaaggtcctagtggagcacggggctgatgtcaacgtgcctgatggcaccggggcacttccaatccatctggcagttcaagagggtcacactgctgtggtcagctttctggcagctgaatctgatctccatcgcagggacgccaggggtctcacacccttggagctggcactgcagagaggggctcaggacctcgtggacatcctgcagggccacatggtggccccgctgtgatctggggtcaccctctccagcaagagaaccccgtggggttatgtatcagaagagaggggaagaaacactttctcttcttgtttctcctgcccactgctgcagtaggggaggagcacagtttgtggcttataggtgttggttttgggggtgtgagtgtttgggggacgtttctcatttgtttttctcactccttttggtgtgttggacagagaagggctcctgcaggccacagccacctaaacggttcagtttcttctgcgcctcaggctgctggggcctcagacgagacccaagggcagagcatttaagagtgaagtcatgacctccagggagcctagaagctggtggccttggccggctgtgctcagagacctgaagtgtgcacgttgcttcaggcatggggggtggggggagcgtcccaaatcaataagaaggtagaatgagttatgagttattcatattctgttggaagcttgttttccagtctcttgtacagcgttttaaaagaaatggattctatttattatgctttattggaaaaaatgttgtaataatttaatgtttttacccattaaattaagacttgtgcatgatcaca

(19) United States
(12) Patent Application Publication
(10) Pub. No.: US 2024/0384347 A1
(43) Pub. Date: Nov. 21, 2024

(54) DETECTING AND TREATING CONDITIONS ASSOCIATED WITH NEURONAL SENESCENCE

(71) Applicant: WAKE FOREST UNIVERSITY HEALTH SCIENCES, Winston-Salem, NC (US)

(72) Inventor: Miranda ORR, Winston Salem, NC (US)

(21) Appl. No.: 18/694,166

(22) PCT Filed: Sep. 23, 2022
(86) PCT No.: PCT/US2022/076916

§ 371 Date: Mar. 21, 2024

Related U.S. Application Data
(60) Provisional application No. 63/261,630, filed on Sep. 24, 2021.

(51) Int. Cl. C12Q 1/6883; A61B 6/03; A61B 6/50; C07K 16/40

(52) U.S. Cl. CPC C12Q 1/6883; A61B 6/037; A61B 6/501; C07K 16/40; C12Q 2600/158

(57) ABSTRACT

Provided herein according to some embodiments is a method of detecting senescent cells and/or neurofibrillary tangles in a subject, comprising: assaying for the expression of CDKN2D in the brain of the subject; and comparing the amount of CDKN2D expression to a control, whereby increased CDKN2D expression relative to the control is indicative of the presence of neurofibrillary tangles in the subject. Methods of treating and monitoring a subject so identified are also provided.

### STATEMENT OF PRIORITY

This application claims the benefit of U.S. Provisional Application Ser. No. 63/261,630, filed Sep. 24, 2021, the entire contents of which are incorporated by reference herein.

### FEDERAL FUNDING

This invention was made with government support under K2BX003804 awarded by the Department of Veterans Affairs and R01 AG068293 awarded by the National Institutes of Health. The government has certain rights in the invention.

### STATEMENT REGARDING ELECTRONIC FILING OF A SEQUENCE LISTING

A Sequence Listing in XML format, entitled 9151-269WO_ST26.xml, 24,123 bytes in size, generated on Sep. 23, 2022, and filed herewith, is hereby incorporated by reference in its entirety for its disclosures.

### BACKGROUND

Cellular senescence is a complex response to cellular stress and/or DNA damage that culminates as a change in cell fate. Senescent cells undergo profound chromatin changes, metabolic dysfunction, cellular remodeling, and resistance to apoptosis¹⁻³. Senescent cell survival contributes to long-term health decline, and senescent cells are notorious for secreting high levels of pro-inflammatory cytokines, chemokines, and extracellular matrix-degrading proteins referred to as senescence-associated secretory phenotype (SASP)⁴⁻⁶. Accumulating data from rodent models indicate that senescent cells contribute to neurodegeneration and cognitive dysfunction with cell types including neurons⁷⁻¹⁰, astrocytes¹¹, microglia¹² and oligodendrocyte precursor cells¹³. Interventions to eliminate senescent cells or reduce their SASP hold great translational appeal for treating many chronic diseases of human aging, including Alzheimer's disease (AD)¹⁴; however, the contribution of cellular senescence to human brain aging and disease remains unknown¹⁵.

Data on the relative proportion of senescent cells in humans have been restricted to accessible tissues. Prior studies using p16^{INK4A}-immunoreactivity as a surrogate marker for senescence reported 5.47%¹⁶ and 3.18%¹⁷ in adipose and 1.95% in epidermal biopsied tissue¹⁷. Identifying senescent cells in patients with or suspected to have AD presents challenges beyond the low abundance of cells and inability to routinely biopsy brain tissue; namely, in vitro-based molecular profiles and senescence assays (i.e., senescence associated beta-galactosidase activity) have generated inconsistent results when applied to the brain⁷. Many types of brain cells secrete molecules that overlap with SASP factors independent of a cell cycle arrest (i.e. glial cells become hyper-proliferative and inflammatory in many neurodegenerative diseases^{18,19}). Moreover, the senescence molecular profile differs across cell types and among tissues^{15,20,21}.

More effective methods of detecting senescent cells in neural tissues is needed.

### SUMMARY

Provided herein according to some embodiments is a method of detecting senescent cells and/or neurofibrillary tangles in a subject, comprising: assaying for the expression of CDKN2D in the brain of the subject; and comparing the amount of CDKN2D expression to a control, whereby increased CDKN2D expression relative to the control is indicative of the presence of neurofibrillary tangles in the subject.

In some embodiments, the method is in vitro and the method comprises obtaining a sample from the subject.

In some embodiments, the sample is cerebral spinal fluid or brain tissue.

In some embodiments, the method is in vivo and the detecting comprises imaging (e.g. PET imaging).

In some embodiments, the method comprises administering a detectable compound (e.g. polynucleotide or antibody) specific for a CDKN2D expressed polynucleotide (e.g., mRNA) or protein (p19INK4D), and further comprising detecting the compound.

Also provided is method of treating a disease associated with the presence of neurofibrillary tangles, comprising administering a treatment for the disease to a subject identified as having neurofibrillary tangles by a method of as taught herein. In some embodiments, the treatment comprises administering a therapeutic as taught herein.

In some embodiments, the disease associated with neurofibrillary tangles is an age-related disease.

In some embodiments, the disease associated with neurofibrillary tangles is a tauopathy.

In some embodiments, the disease is selected from mild cognitive impairment, Alzheimer's disease, traumatic brain injury, primary age-related tauopathy (PART), neurofibrillary tangle-predominant dementia (NFTPD), Pick disease, Parkinson's disease, Chronic traumatic encephalopathy (CTE), progressive supranuclear palsy (PSP), frontotemporal dementia, frontotemporal lobar degeneration, progressive supranuclear palsy, corticobasal degeneration, Amyotrophic Lateral Sclerosis (ALS), and Huntington's Disease.

In some embodiments, the administering is by direct administration to the brain of the subject.

In some embodiments, the treating comprises inhibiting the formation of, or reducing the presence of, neurofibrillary tangles in the subject.

In some embodiments, the treating inhibits the expression or activity of CDKN2D or p19INK4D.

In some embodiments, the treating inhibits cellular senescence caused by or associated with neurofibrillary tangles in a subject.

In some embodiments, the treatment comprises a genetic modifying agent, antibody or fragment thereof. In some embodiments, the genetic modifying agent comprises an antisense oligonucleotide, an RNAi, an siRNA, or a gene editing system selected from a CRISPR system, a zinc finger nuclease system, and a TALE system.

In some embodiments, the treatment comprises a therapeutic antibody or fragment thereof that specifically binds to the protein encoded by CDKN2D.

In some embodiments, the genetic modifying agent, antibody or fragment thereof comprises a detectible group.

In some embodiments, the method includes performing PET imaging on the subject.

Further provided is a method of monitoring the progress of a neurofibrillary-associated disease in a subject comprising: detecting a first level of CDKN2D expression in a biological sample obtained from the subject at a first time point; detecting a second level of CDKN2D expression in a biological sample obtained from the subject at a second time point; and comparing the second level of CDKN2D expression with the first level of CDKN2D expression, wherein said comparison indicates the progress of the neurofibrillary-associated disease in the subject. In some embodiments, the first time point is a time point before initiation of a treatment regimen, and wherein the second time point is a time point after initiation of a treatment regimen.

Also provided is the use of a therapeutic as taught herein in a method of treating a disease associated with the presence of neurofibrillary tangles, or in the manufacture of a medicament for treating a disease associated with the presence of neurofibrillary tangles, in a subject identified as having neurofibrillary tangles by a method of as taught herein.

### BRIEF DESCRIPTION OF THE DRAWINGS

FIG. 1A-1K: The prominent senescent cell type in the dorsal frontal forebrain were excitatory neurons. Eigengenes for each gene list (a, d, h) canonical senescence pathway (CSP); (b, e, i) senescence initiating pathway (SIP); and (c, f, j) senescence response pathway (SRP) were computed using principal component analyses. (a-c) The proportion of cells from each brain expressing the respective eigengene were plotted. (d-f) Cell types and (g) counts represented in the senescent cell population discovered in a-c. (h-j) The ratio of senescent excitatory neurons to total neurons that expressed the respective eigengenes within each brain. (j) Scatter plot for the ratio of senescent excitatory neurons to the total number of excitatory neurons in cohort 1 brains. Each dot represents one brain. The size of the dots depicts the ratio in SRP. The senescence excitatory ratios of CSP highly correlated with SIP (Pearson correlation: 0.96) and SRP (0.90). Also, the SIP ratio was positively correlated with the SRP ratio (0.93). n=48 brains. The yellow (a-c) and black (g-k) dots represent outlier samples, the lower and upper hinges of box plots correspond to the first and third quartiles. The middle line shows the median. Cell populations: astrocytes [Ast], endothelial cells [End], excitatory neurons [Ex], inhibitory neuron [In], microglia [Mic], oligodendrocytes [Oli], oligodendrocyte precursor cells [Opc], and pericytes [Per]) were classified as described in Sharpless and Sherr, Nat Rev Cancer. 2015 July; 15(7):397-408; doi: 10.1038/nrc3960.

FIG. 2A-2F: Excitatory neuron neurofibrillary tangle eigengene expression significantly correlated with senescence expression. (a) Eigengenes representing neurofibrillary tangle (NFT) expression were calculated from two separate datasets, Dunckley32 and (b) Garcia36, respectively. Cell types (a, b) and counts (c) expressing each NFT eigengene were calculated and plotted. (d-e) The ratio of NFT-containing excitatory neurons to total neurons expressing each respective eigengene within each brain. The black dots represent outlier samples, the lower and upper hinges of box plots correspond to the first and third quartiles. The middle line shows the median. (f) Scatter plot for eigengene values for CSP genes on x axis versus Dunckley NFT marker genes on y axis. Each dot represents one neuron. Red line: intercept; Blue line: best linear fit, standard error=0.002, R2=0.6822, p<2e-16.

FIG. 3A-3F: Senescent excitatory neurons contain NFTs and NFT-bearing neurons are senescent. (a-c) Plots of total neuron counts, pink, against expression of the eigengene CSP, (b) SIP or (c) SRP. Cell densities of where the NFT-bearing neurons (green) lie within the plot (inset). (d-f) Plots of total neuron counts, pink, against expression of the NFTDunckley eigengene. Cell densities of where the CSP, (d) SRP or (e) SIP cell populations lie within the plot (inset). Larger plots are scaled by the number of cells and insets are scaled by cell density. Mean and standard deviation (sd) are calculated for the eigengene value of all neurons.

FIG. 4A-4I: Upregulated CDKN2D and p19INK4D deposition co-occur with tau neuropathology in human Alzheimer's disease. (a) Weight of each gene in the canonical senescence pathway (CSP) eigengene based on principle component analysis; CDKN2D had the highest weight. (b-e) Expression of CDKN2D protein product, p19, was determined using immunohistochemistry. Frontal cortex in a control (b) and Alzheimer's disease neuropathologic change (ADNC) cases (c-e). (f-i) Frontal cortex of the same cases were immunostained with AT8 (phosphorylated tau) (i.e. corresponding AT8 stains are directly below the p19 stains). The control PART case (b and f) displayed no immunoreactivity for either p19 or AT8. (c) Diffuse p19 cytoplasmic staining patterns were observed in several AD cases. (d) Neuritic plaques observed in intermediate and high ADNC were immunoreactive to both p19 and (h) AT8. (e) Intranuclear p19 was observed in two cases with a history of head injury. (i) Dense AT8 immunoreactive with neurofibrillary tangle (NFT) pathology co-occurred with nuclear p19 in subject “e”. All microscopic images were taken at 20×, and the scale bars represent 40 μm.

FIG. 5A-5C: Relative proportion of senescent cells to total cellular population in the dorsal frontal forebrain in cohort 2. Probability density for ratio of senescent cells in (a) CSP, (b) SIP and (c) SRP in cohort 2. The details of boxplots are the same as FIG. 1. In both cohorts the ratio of senescent cells varies between 0-15%.

FIG. 6: Prominent senescent cell types in the dorsal lateral prefrontal cortex in cohort 2. The cutoff definition and abbreviations are similar to FIG. 1.

FIG. 7A-7D: Relative proportion of senescent excitatory neurons to total neuronal population in cohort 2. Probability density for ratio of senescent neurons in (a) CSP, (b) SIR and (c) SRP in cohort 2. The black dots show the outlier samples, the lower and upper hinges of box plots correspond to the first and third quartiles. The middle line shows the median. The ratio of senescent excitatory neurons approximately varies between 0-30% in cohort 2. (d) Scatter plot for the ratio of senescent excitatory neurons to the total number of excitatory neurons in 28 brains. Each dot represents one brain. The size of the dots shows this ratio in SRP. The senescence excitatory ratio of CSP highly correlates with SIP (0.94), its correlation with SRP ratio is 0.38. The correlation between SIP and SRP ratios is 0.46.

FIG. 8A-8B8: Excitatory neurons are the prominent NFT cell type in the dorsal frontal forebrain in cohort 2. The snRNA-Seq transcriptomic data was analyzed for cell populations expressing the eigengene using (a) Dunckley gene list or (b) Garcia gene list. The cutoff definition and abbreviations are similar to FIG. 1.

FIG. 9A-9B: The ratio of senescent neurons based on two separate eigengenes in cohort 2. (a) The proportion of senescent excitatory neurons to total neurons expressing the Dunkley and (b) Garcia NFT eigengenes. The black dots represent outlier samples, the lower and upper hinges of box plots correspond to the first and third quartiles. The middle line shows the median.

FIG. 10A-10B: Weight of each gene in the SIP (a) and SRP (b) eigengene based on our principle component analysis.

FIG. 11A-11B: Excitatory neurons are the prominent senescent cells based on CDKN2D in (a) cohort 1 and (b) cohort 2. The cutoff definition and abbreviations are similar to FIG. 1.

### DETAILED DESCRIPTION

According to the present invention, compositions and methods useful to predict, detect and/or treat Alzheimer's disease (AD), dementia, other age-related diseases and neuronal senescence are provided.

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et al. Current Protocols in Molecular Biology (Green Publishing Associates, Inc. and John Wiley & Sons, Inc., New York).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As described further below, it has been found that expression of cell cycle inhibitor, CDKN2D/p19, contributes most to the senescence profile in neural tissues, and p19 protein is expressed within AD neural tissue. Moreover, the senescent excitatory neuron population significantly overlaps with neurons expressing transcriptomes consistent with neurofibrillary tangle (NFT) pathology (−log10(p)=1336), the closest correlate with neurodegeneration and dementia in AD. Excitatory neurons constitute a prominent senescent cell population in human brain and reveal a novel molecular regulator overlapping between senescence and AD pathogenesis, CDKN2D/p19.

As used in this document, CDKN2D indicates the cyclin dependent kinase inhibitor 2D gene, while the resulting protein or fragment thereof is referred to as p19 or p19INK4D. With the accumulation of senescent cells with age and the connection between that accumulation, dementia and other age-related diseases, detecting and/or controlling the expression of CDKN2D/p19INK4D can contribute to effective treatment.

The term neurofibrillary tangles (NFT) is known to those skilled in the art and refers to aggregate tangles that are mainly composed of aggregates of highly phosphorylated tau protein.

The term “fragment,” as applied to a protein, will be understood to mean an amino acid sequence of reduced length relative to a reference protein (e.g., wild-type p19INK4D protein) or amino acid sequence and having contiguous amino acids identical to the reference protein.

The terms “polynucleotide”, “nucleic acid,” “nucleic acid molecule,” and “oligonucleotide” are used interchangeably and refer to a polymeric form of nucleotides of any length, either deoxyribonucleotides or ribonucleotides or analogs thereof. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

As used herein, “expression” refers to the process by which a gene such as CDKN2D is transcribed into mRNA and/or the process by which the transcribed mRNA is subsequently translated into a protein, such as p19INK4D. If the polynucleotide is derived from genomic DNA, expression may include splicing of the mRNA in a eukaryotic cell.

“Pharmaceutically acceptable carrier” (sometimes referred to as a “carrier”) refers to a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

A “control” as used herein may refer to a sample or measured value obtained from one or more individuals know to be free of conditions associated with neurofibrillary tangles and/or neuronal senescence, for example, individuals younger than about 30, 25, 20, 19, 18, 17, 16, 15 years of age. A control may also be a control of an age-matched individual or population that is cognitively normal, which may comprise averaging a value of a cognitively normal population of individuals. Design of standard controls is well-known in the art, and control values may be utilized rather than a sample control. Reference values can be as established in the art and/or may be measured in an individual or a population of individuals characterised as having or without a particular diagnosis, prediction and/or prognosis, for example, of the NFT-associated disease or condition or neuronal senescence-associated disease or condition.

A disease or disorder associated with neurofibrillary tangles refers to any disease or disorder that is caused by or has an association with, or has at least one symptom caused by or has an association with, neurofibrillary tangles (NFT), which may include an increase in CDKN2D expression. Examples include, but are not limited to, Parkinson's disease, Alzheimer's disease, prion disease, chronic traumatic encephalopathy (CTE), multisystem proteinopathy (MSP), Guam Parkinson-dementia complex (G-PDC) and ALS (G-ALS), facial onset sensory and motor neuronopathy, primary lateral sclerosis (PLS), progressive muscular atrophy (PMA), frontotemporal dementia, Perry disease, and others. A disease associated with neurofibrillary tangles may also be identified by assays as described herein, including with a detectably labeled p19INK4D-binding compound. Neurofibrillary tangles are thought to be formed by hyperphosphorylated tau protein fibers, typically a misfolded protein formation, that form in the neuron and aggregate into a mass. NFTs may comprise one or more maturities. For example, the NFT may include pretangles, which are found in morphologically normal neurons with a healthy nucleus and are typically diffusely scattered abnormal fibers. Mature tangles are also encompassed within the definition of NFTs, and typically found in neurons with a shrunken nucleus and take on the shape of the cell in which they reside. Ghost tangles are more advanced and more loosely bundled than mature tangle. NFT as utilized herein encompasses all tangle maturity levels, including pretangles, mature tangles, and ghost tangles. See, e.g., Moloney et al., Alzheimer's Dement. 2021; 17:1554-1574, doi: 10.1002/alz.12321.

A “subject” may be any vertebrate organism in various embodiments. A subject may be individual to whom an agent is administered, e.g., for experimental, diagnostic, and/or therapeutic purposes, or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult or geriatric subject. In some embodiments a human subject is at least 50, 60, 70, 80, or 90 years old.

“Treat,” “treating” and similar terms as used herein in the context of treating a subject refer to providing medical and/or surgical management of a subject. As used herein, the terms “treating” or “treatment” or “to treat” or “alleviating” or “to alleviate” refer to therapeutic measures that cure, reduce, slow down, lessen symptoms of, decrease and/or halt progression of a diagnosed disease or neurofibrillary tangle associated disorder or senescence associated disorder, and prophylactic or preventative measures that prevent or slow the development of said diseases or disorders.

Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

Grammatical variations of “administer,” “administration,” and “administering” to a subject include any route of introducing or delivering to a subject a therapeutic. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like.

“Concurrent administration,” “administration in combination,” “simultaneous administration,” or “administered simultaneously” as used herein, means that the therapeutics are administered at the same point in time, overlapping in time, or one following the other. In the latter case, the two therapeutics are administered at times sufficiently close that the results observed are indistinguishable from those achieved when they are administered at the same point in time. “Systemic administration” refers to the introducing or delivering to a subject an agent via a route which introduces or delivers the therapeutic to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

The present invention is based, in part, on the discovery that the expression level of the cyclin dependent kinase inhibitor 2D (CDKN2D) gene and/or protein encoded by the gene, p19INK4D, is associated with senescent neurofibrillary tangle (NFT)-bearing neurons. CDKN2D encodes the p19INK4D protein, which is a cyclin-dependent kinase inhibitor that can form a stable complex with CDK4 or CDK6. See, e.g., Kalus et al., NMR structural characterization of the CDK inhibitor p19INK4d. FEBS Lett 1997; 401:127-32; doi: 10.1016/S0014-5793(96)01465-2.

Accordingly, one aspect of the invention relates to detecting neurofibrillary tangles in a subject comprising assaying for CDKN2D gene expression and/or expression levels of the CDKN2D encoded protein p19INK4D. In an embodiment, the method includes comparing the amount of CDKN2D or p19INK4D to a control, whereby increased CDKN2D or p19INK4D expression relative to the control is indicative of the presence of neurofibrillary tangles. In some embodiments, detecting comprises detecting the expression in a subcellular compartment (e.g., the cytosol) of a cell such as an excitatory neuron.

In some embodiments, the CDKN2D is a mammalian CDKN2D, such as mouse or human, or a functional fragment thereof. In an embodiment, the CDKN2D encodes a p19INK4D polypeptide, or a fragment thereof. Example CDKN2D polynucleotides and the encoded p19INK4D polypeptides are provided in Table 1.

TABLE 1

| | | SEQ |
|---|---|---|
| | | ID |
| Description | Sequence | NO: |
| Mus musculus | agagcagaccccgcccgcgcaaggaaaggagggagguccaccgguauccacuaugcuucuggaagaagu | 1 |
| cyclin dependent | cugcgucggcgaccgguugaguggcgcagcggcccguggcgacgugcaagagguccgccgccuucuucg | |
| kinase inhibitor | gaaguccagcaguugcuuuggagcuccugaagcaaggugccagccccaauguccaagaugecuccggua | |
| 2D (Cdkn2d), | cuaguccugugcaugaugcggcucgcaccggguuccuggacacccugaagguucugguggagcauggug | |
| transcript variant | cugaugucaaugcccuggacagcacugggucgcuccccauccaucuggcgauaagagagggccauagcu | |
| 2, mRNA | ccguggucagcuuccuagcuccugaaucugaucuccaccacagggaccuuccggucucacuccccugga | |
| | guuggcucggcagagaggggcucagaaccucauggacauucugcaggggcacaugaugaucccaaugug | |
| | acccaaggccacugucuccagccuuacuggguuacuugucaacaaaagaggaaagaaacuuucucuagg | |
| | ggcuccuacaggcaacagccaucuaaacgguucaguuuccucugcacuggggcugcaccagggcagggg | |
| | uuuaaagcccuagccucagagugaggucaucacuucccgggccccuuggaagcuggugaccuuggcagg | |
| | cugugcucagagagcccugaagugugagccaucuacuuugggcaugugagggggaagggaaagccagag | |
| | uuugaaauauaaauauuuauugcuuuauggaaa | |
| Mus musculus | MLLEEVCVGDRLSGAAARGDVQEVRRLLHRELVHPDALNRFGKTALQVMMFGSPAVALELLKQGASPNV | 2 |
| (Cdkn2d), | QDASGTSPVHDAARTGFLDTLKVLVEHGADVNALDSTGSLPIHLAIREGHSSVVSFLAPESDLHHRDAS | |
| transcript variant | GLTPLELARQRGAQNLMDILQGHMMIPM | |
| 2, amino acid | | |
| Mus musculus | agagcagaccccgcccgcgcaaggaaaggagggagguccaccgguauccacuaugcuucuggaagaagu | 3 |
| (Cdkn2d), | accgggagcuggugcauccugacgcccugaaccgcuuuggcaagacggccuugcaggugcccucuugaa | |
| transcript variant | ggaucucugguuauuucagggacaucuucauuucggcaggucaugauguuuggaaguccagcaguugcu | |
| 3, mRNA | uuggagcuccugaagcaaggugccagccccaauguccaagaugccuccgguacuaguccugugcaugau | |
| | gcggcucgcaccggguuccuggacacccugaagguucugguggagcauggugcugaugucaaugcccug | |
| | gacagcacugggucgcuccccauccaucuggcgauaagagagggccauagcuccguggucagcuuccua | |
| | gcuccugaaucugaucuccaccacagggacgcuuccggucucacuccccuggaguuggcucggcagaga | |
| | ggggcucagaaccucauggacauucugcaggggcacaugaugaucccaaugugacccaaggccacuguc | |
| | uccagccuuacuggguuacuugucaacaaaagaggaaagaaacuuucucuuuucacaccuguccauuga | |
| | agaagggagugggaggagcaguuugugguuuauugguguugauuucuugaguguguguguuuggggggu | |
| | guuucucauuuguuuuucucaccccuuuugguguguuggacaaagaaggggcuccuacaggcaacagcc | |
| | aucuaaacgguucaguuuccucugcacugcgggcugcaccagopgcagcgguuuaaagcccuagccuca | |
| | gagugaggucaucacuucccgggccccuuggaagcuggugaccuuggcaggcugugcucagagagcccu | |
| | gaagugugagccaucuacuuugggcaugugagggggaagggaaagcauuucaaaucaauuaaaaggaua | |
| | acaugaguucauuuuucuuucuuggaagguuuccagucuguuguacagaguuugaaauauaaauauuua | |
| | uugcuuuauggaaa | |
| Mus musculus | MMFGSPAVALELLKQGASPNVQDASGTSPVHDAARTGFLDTLKVLVEHGADVNALDSTGSLPIHLAIRE | 4 |
| (Cdkn2d), | GHSSVVSFLAPESDLHHRDASGLTPLELARQRGAQNLMDILQGHMMIPM | |
| transcript variant | | |
| 3, amino acid | | |
| Mus musculus | agcugcgagcgcugcggucguacgggcggagcgcgcaggcucugaggcccgcgcaaaugaucauagagu | 5 |
| (Cdkn2d), | uggcccugguggcaccgcagucccuagaguucugauccagcucuugcugguuccccagcccugaccuua | |
| transcript variant | acugggcuuggggcugcgugcguuucacaguccaccgguauccacuaugcuucuggaagaagucugcgu | |
| 4, mRNA | cggcgaccgguugaguggcgcagcggcccguggcgacgugcaagagguccgccgccuucuucaccggga | |
| | gcuggugcauccugacgcccugaaccgcuuuggcaagacggccuugcaggugcccucuugaaggaucuc | |
| | ugguuauuucagggacaucuucauuucggcaggucaugauguuuggaaguccagcaguugcuuuggagc | |
| | uccugaagcaaggugccagccccaauguccaagaugcccuccgguacuaguccugugcaugaugcggcu | |
| | cgcacccggguuccuggacacccugaagguucugguggagcauggugcugaugucaaugcccuggacag | |
| | cacugggucgcuccccauccaucuggccgauaagagagggccauagcuccguggucagcuuccuagcuc | |
| | cugaaucugaucuccaccacagggacgcuuccggucucacuccccuggaguuggcucggcagagagggg | |
| | cucagaaccucauggacauucugcagcgggcacaugaugaucccaaugugacccaaggccacugucucc | |
| | agccuuacugggunacuugucaacaaaagaggaaagaaacuuucucuuuucacaccuguccauugaaga | |
| | agggagugggaggagcaguuugugguuuauugguguugauuucuugaguguguguguuugggggguguu | |
| | ucucauuuguuuuucucacccccuuuugguguguuggacaaagaaggggcuccuacaggcaacagccau | |
| | cuaaacgguucaguuuccucugcacuggggcugcaccagcggcagggguuuaaagcccuagccucagag | |
| | ugaggucaucacuucccgggccccuuggaagcuggugaccuuggcaggcugugcucagagagcccugaa | |
| | gugugagccaucuacuuugggcaugugagcgcggaagcgaaagcauuucaaaucaauuaaaaggauaac | |
| | augaguucauuuuucuuucuuggaagguuuccagucuguuguacagaguuugaaauauaaauauuuauu | |
| | gcuuuauggaaa | |
| Mus musculus | MMFGSPAVALELLKQGASPNVQDASGTSPVHDAARTGFLDTLKVLVEHGADVNALDSTGSLPIHLAIRE | 6 |
| (Cdkn2d), | GHSSVVSFLAPESDLHHRDASGLTPLELARQRGAQNLMDILQGHMMIPM | |
| transcript variant | | |
| 4, amino acid | | |
| Homo sapiens | agagcagaccccgcccggcgaggaggagggagggugaguuagggggagacccggcccccaaggggcggg | 7 |
| cyclin dependent | cgccgggcagggccccgcgggggccgaggguugggcccggcucccagccccucgccguccuccggcuga | |
| kinase inhibitor | cagggggaggagcccgccgggagggccggggucucgggcuggggagccgggacgggagagcagcgcagc | |
| 2D (CDKN2D), | cgggugcaccgcggccgcgccccgggagggcuguucgggccagcgcccgccggcugcuccggcugacag | |
| transcript variant | cgccgggcugggggcggggcggggggcuuugcaggccgccagugucgacaugcugcuggaggagguucg | |
| 1, mRNA | cgccggcgaccggcugaguggggcggcggcccggggcgacgugcaggaggugcgccgccuucugcaccg | |
| | ggagcuggugcaucccgacgcccucaaccgcuucggcaagacggcgcugcaggucaugauguuuggcag | |
| | caccgccaucgcccuggagcugcugaagcaaggugccagccccaauguccaggacaccuccgguaccag | |
| | uccaguccaugacgcagcccgcacuggauuccuggacacccugaagguccuaguggagcacggggcuga | |
| | ugucaacgugccugauggcaccggggcacuuccaauccaucuggcaguucaagagggucacacugcugu | |
| | ggucagcuuucuggcagcugaaucugaucuccaucgcagggacgccaggggucucacacccuuggagcu | |
| | ggcacugcagagaggggcucaggaccucguggacauccugcagggccacaugguggccccgcugugauc | |
| | uggggucacccucuccagcaagagaaccccgugggguuauguaucagaagagaggggaagaaauguugg | |
| | acagagaagggcuccugcaggccacagccaccuaaacgguucaguuucuucuggccucaggcugcuggg | |
| | gccucagacgagacccaagggcagagcauuuaagagugaagucaugaccuccagggagccuagaagcug | |
| | guggccuuggccggcugugcucagagaccugaagugugcacguugcuucaggcauggggggugggggga | |
| | gcgucccaaaucaauaagaagguagaaugaguuaugaguuauucauauucuguuggaagcuuguuuucc | |
| | agucucuuguacagcguuuuaaaagaaauggauucuauuuauuaugcuuuauuggaaaaaauguuguaa | |
| | uaauuuaauguuuuuacccauuaaauuaagacuugugcaugaucaca | |
| Homo sapiens | agagcagaccccgcccggcgaggaggagggagggccgccagugucgacaugcugcuggaggagguucgc | 8 |
| cyclin dependent | gccggcgaccggcugagugggggcggcccccggggcgacgugcaggaggugcgccgccuucugcaccgc | |
| kinase inhibitor | ggagcuggugcaucccgacgcccucaaccgcuucggcaagacggcgcugcaggucaugauguuuggcag | |
| 2D (CDKN2D), | caccgccaucgcccuggagcugcugaagcaaggugccagccccaauguccaggacaccuccgguaccag | |
| transcript variant | uccaguccaugacgcagcccgcacuggauuccuggacacccugaagguccuaguggagcacggggcuga | |
| 2, mRNA | ugucaacgugccugauggcaccgcggcacuuccaauccaucuggcaguucaagagcggucacacugcug | |
| | uggucagcuuucuggcagcugaaucugaucuccaucgcagggacgccagcggucucacacccuuggagc | |
| | uggcacugcagagagcggcucaggaccucguggacauccugcagggccacaugguggccccgcugugau | |
| | cuggggucacccucuccagcaagagaacccccgugggguuauguaucagaagagaggggaagaaacacu | |
| | uucucuucuuguuucuccugcccacugcugcaguagccggaggagcacaguuuguggcuuauagguguu | |
| | gguuuugggggugugaguguuugggggacguuucucauuuguuuuucucacuccuuuugguguguugga | |
| | cagagaagggcuccugcaggccacagccaccuaaacgguucaguuucuucugcgcccucaggcugcugg | |
| | ggccucagacgagacccaagggcagagcauuuaagagugaagucaugaccuccagggagccuagaagcu | |
| | gguggccuuggccggcugugcucagagaccugaagugugcacguugcuucaggcaugccgccgugccgg | |
| | gagcgucccaaaucaauaagaagguagaaugaguuaugaguuauucauauucuguuggaagcuuguuuu | |
| | uccagucucuuguacagcguuuuaaaagaaauggauucuauuuauuaugcuuuauuggaaaaaauguug | |
| | uaauaauuuaauguuuuuacccauuaaauuaagacuugugcaugaucaca | |
| Homo sapiens | MLLEEVRAGDRLSGAAARGDVQEVRRLLHRELVHPDALNRFGKTALQVMMFGSTAIALELLKQGASPNV | 9 |
| cyclin dependent | QDTSGTSPVHDAARTGFLDTLKVLVEHGADVNVPDGTGALPIHLAVQEGHTAVVSFLAAESDLHRRDAR | |
| kinase inhibitor | GLTPLELALQRGAQDLVDILQGHMVAPL | |
| 2D (CDKN2D), | | |
| amino acid | | |
| Mus musculus | agcugcgagcgcuggggucguacgggcggagggcgcaggcucugaggccggcaaaugaucauagaguug | 10 |
| cyclin dependent | gcccugguggcaccgcagucccuagaguucugauccagcucuugcugguuccccagcccugaccuuaac | |
| kinase inhibitor | ugggcuuggggcuggguggguuucacaguccaccgguauccacuaugcuucuggaagaagucugcgucg | |
| 2D (Cdkn2d), | gcgaccgguugaguggcgcagcggcccguggcgacgugcaagagguccgccgccuucuucaccgggagc | |
| transcript variant | uggugcauccugacgcccugaaccgcuuuggcaagacggccuugcaggucaugauguuuggaaguccag | |
| 1 | caguugcuuuggagcuccugaagcaaggugccagccccaauguccaagaugccuccgguacuaguccug | |
| | ugcaugaugcggcucgcaccggguuccuggacacccugaagguucugguggagcauggugcugauguca | |
| | augcccuggacagcacugggucgcuccccauccaucuggcgauaagagagggccauagcuccgugguca | |
| | gcuuccuagcuccugaaucugaucuccaccacagggacgcuuccggucucacuccccuggaguuggcuc | |
| | ggcagagaggggcucagaaccucauggacauucugcaggggcacaugaugaucccaaugugacccaagg | |
| | ccacugucuccagccuuacuggguuacuugucaacaaaagaggaaagaaacuuucucuuuucacaccug | |
| | uccauugaagaagggagugggaggagcaguuugugguuuuugguguugauguuggacaaagaaggggcu | |
| | ccuacaggcaacagccaucuaaacgguucaguuuccucugcacuggggcugcaccagggcagggguuua | |
| | aagcccuagccucagagugaggucaucacuucccgggccccuuggaagcuggugaccuuggcaggcugu | |
| | gcucagagagcccugaagugugagccaucuacuuugggcaugugagggggaagggaaagcauuucaaau | |
| | caauuaaaaggauaacaugaguucauuuuuuuucuuggaagguuuccagucuguuguacagaguuugaa | |
| | aaauauaaauugcuuuauggaaa | |
| Homo sapiens | AGAGCAGACCCCGCCCGGCGAGGAGGAGGGAGGGTGAGTTAGGGGGAGACCCGGCCCCCAAGGGGCGGG | 11 |
| isolate CHM13 | CGCCGGGCAGGGCCCCGCGGGCGGCCGAGGGTTGGGCCCGGCTCCCAGCCCCTCGCCGTCCTCCGGCTG | |
| chromosome 19, | ACAGGGGGAGGAGCCCGCCGGGAGGGCCGGGGTCTCGGGCTGGGGAGCCGGGACGGGAGAGCAGCGCAG | |
| alternate assembly | CCGGGTGCACCGCGGCCGCGCCCCGGGAGGGCTGTTCGGGCCAGCGCCCGCCGGCTGCTCCGCGCTGAC | |
| T2T-CHM13v2.0, | AGCGCCGGGCTGGGGCGGGGCGGGGGGCTTTGCAGGCCGCCAGTGTCGACATGCTGCTGGAGGAGGTTC | |
| Genome accession | GCGCCGGCGACCGGCTGAGTGGGGCGGCGGCCCGGGGCGACGTGCAGGAGGTGCGCCGCCTTCTGCACC | |
| NC_060943 | GCGAGCTGGTGCATCCCGACGCCCTCAACCGCTTCGGCAAGACGGCGCTGCAGGTGAGGCCGGGCCGGT | |
| | CCAGGTTGGGGGGGGGGGGCTAAGCGGGATGAGGATCGAGACCCCAGAGCTCCTCTCTGGTCTCCTGGC | |
| | CCTGGTGGTGACCCACTGGGAACCGGTCCTTCTCTAGGCTTTCATTGTTCCCCCACAGTCCCCCTTCGA | |
| | AGGTGGGTGGTGGGTGAGGCTAGGTTGTGGGGGGAGGGGAGAGCTTTTTAGCATCTTCTCTGGGAGGAT | |
| | TCCTGGTTCTCTGAGTGCCGGGGATTCACTTTCCCTGGGGAGGTTCTTATTCCGTGGGTGCGGGTTCAG | |
| | CTCCTTTGGATGCCGTTTCCATGGGGACGGCTCACGTTTCTTGGGGCTTCTGTGGGGAGATCCGATCTC | |
| | TGGAAGGAGAGGTCCTGTCTCTAGGGGTTCCTCATTTTGTAGGGGGCTCTCTGAGACAAATCTGGTTAC | |
| | TTGGGGGTGGGGTCTTCATTCCTAAAGGTTATGCGGATCCTTGATATAGTATGGGGGCCCCAATGGAGG | |
| | AACAATGTCAGATTGCAAGACATCCCTATATTCCCTGGGAGACATTTTACAGGGGAGTCTCGTTTCTCT | |
| | TTTATTATTACTTTTGGAATCCCGCTTCTTGAAAGGAGCTCTCAGGTACTCTGAGGTGACATCACCATT | |
| | ATGTAGGAGGAGGCTATTTCCTGGAGAAGGGGTTCGCTTTCCTTTGACATCTTTTTTCCCTTATGAGGA | |
| | CTATCAATTCTTTGGGGGTAGTTCTCCATCTCTGGGGGCCCATTTCCTGGAGGGGATTTTCTGTTTCCT | |
| | GGTGATTGCCCATATCTGGGGGCGTCTTTGGTCCCTTGGGGGAGAGTCTTAGGTTCCTTGAAAGTTTCC | |
| | ATGGGGGGCTGTTCTCTGGGTTGGGGGGTCTCATTCTTAAGGGAGTGACCAGCTTTTGGGGGGGGTCCC | |
| | TATTCCCAGAAGAACTTTCCTGGGGGCTTATGCAGCTTCTCCGTTCACTAGAGGACCCCTCCTCTAGCA | |
| | TTTCCTGTTTCTGGGAGATGCCTCTTTCTGGATGACCCTTCTCTTTCCAGTGACCCCTTTTGTGGAACC | |
| | TCCTCCCTTGAGGACCTCCTGATCCTCTGTCCCTCACACAGGTCATGATGTTTGGCAGCACCGCCATCG | |
| | CCCTGGAGCTGCTGAAGCAAGGTGCCAGCCCCAATGTCCAGGACACCTCCGGTACCAGTCCAGTCCATG | |
| | ACGCAGCCCGCACTGGATTCCTGGACACCCTGAAGGTCCTAGTGGAGCACGGGGCTGATGTCAACGTGC | |
| | CTGATGGCACCGGGGCACTTCCAATCCATCTGGCAGTTCAAGAGGGTCACACTGCTGTGGTCAGCTTTC | |
| | TGGCAGCTGAATCTGATCTCCATCGCAGGGACGCCAGGGGTCTCACACCCTTGGAGCTGGCACTGCAGA | |
| | GAGGGGCTCAGGACCTCGTGGACATCCTGCAGGGCCACATGGTGGCCCCGCTGTGATCTGGGGTCACCC | |
| | TCTCCAGCAAGAGAACCCCGTGGGGTTATGTATCAGAAGAGAGGGGAAGAAACACTTTCTCTTCTTGTT | |
| | TCTCCTGCCCACTGCTGCAGTAGGGGAGGAGCACAGTTTGTGGCTTATAGGTGTTGGTTTTGGGGGTGT | |
| | GAGTGTTTGGGGGACGTTTCTCATTTGTTTTTCTCACTCCTTTTGGTGTGTTGGACAGAGAAGGGCTCC | |
| | TGCAGGCCACAGCCACCTAAACGGTTCAGTTTCTTCTGCGCCTCAGGCTGCTGGGGCCTCAGACGAGAC | |
| | CCAAGGGCAGAGCATTTAAGAGTGAAGTCATGACCTCCAGGGAGCCTAGAAGCTGGTGGCCTTGGCCGG | |
| | CTGTGCTCAGAGACCTGAAGTGTGCACGTTGCTTCAGGCATGGGGGGTGGGGGGAGCGTCCCAAATCAA | |
| | TAAGAAGGTAGAATGAGTTATGAGTTATTCATATTCTGTTGGAAGCTTGTTTTCCAGTCTCTTGTACAG | |
| | CGTTTTAAAAGAAATGGATTCTATTTATTATGCTTTATTGGAAAAAATGTTGTAATAATTTAATGTTTT | |
| | TACCCATTAAATTAAGACTTGTGCATGATCACA | |
| Homo sapiens | AGAGCAGACCCCGCCCGGCGAGGAGGAGGGAGGGTGAGTTAGGGGGAGACCCGGCCCCCAAGGGGCGGG | 12 |
| chromosome 19, | CGCCGGGCAGGGCCCCGCGGGCGGCCGAGGGTTGGGCCCGGCTCCCAGCCCCTCGCCGTCCTCCGGCTG | |
| GRCh38.p14 | ACAGGGGGAGGAGCCCGCCGGGAGGGCCGGGGTCTCGGGCTGGGGAGCCGGGACGGGAGAGCAGCGCAG | |
| Primary Assembly; | CCGGGTGCACCGCGGCCGCGCCCCGGGAGGGCTGTTCGGGCCAGCGCCCGCCGGCTGCTCCGCGCTGAC | |
| Genome Accession | AGCGCCGGGCTGGGGCGGGGCGGGGGGCTTTGCAGGCCGCCAGTGTCGACATGCTGCTGGAGGAGGTTC | |
| NC_000019 | GCGCCGGCGACCGGCTGAGTGGGGCGGCGGCCCGGGGCGACGTGCAGGAGGTGCGCCGCCTTCTGCACC | |
| | GGGAGCTGGTGCATCCCGACGCCCTCAACCGCTTCGGCAAGACGGCGCTGCAGGTGAGGCCGGGCCGGT | |
| | CCAGGTTGGGGGGGGGGGGCTAAGCGGGATGAGGATCGAGACCCCAGAGCTCCTCTCTGGTCTCCTGGC | |
| | CCTGGTGGTGACCCACTGGGAACCGGTCCTTCTTCTCTAGGCTTTCATTGTTCCCCCACAGTCCCCCTT | |
| | CGAAGGTGGGTGGTGGGTGAGGCTAGGTTGTGGGGGGAGGGGAGAGCTTTTTAGCATCTTCTCTGGGAG | |
| | GATTCCTGGTTCTCTGAGTGCCGGGGATTCACTTTCCCTGGGGAGGTTCTTATTCCGTGGGTGCGGGTT | |
| | CAGCTCCTTTGGATGCCGTTTCCATGGGGACGGCTCACGTTTCTTGGGGCTTCTATGGGGAGATCCGAT | |
| | CTCTGGAAGGAGAGGTCCTGTCTCTAGGGGTTCCTCATTTTGTAGGGGGCTCTCTGAGACAAATCTGGT | |
| | TACTTGGGGGTGGGGTCTTCATTCCTAAAGGTTATGCGGATCCTTGATATAGTATGGGGGCCCCAATGG | |
| | AGGAACAATGTCAGATTGCAAGACATCCCTATATTCCCTGGGAGACATTTTACAGGGAAGTCTCGTTTC | |
| | TCTTTTATTATTACTTTTGGAATCCCGCTTCTTGAAAGGAGCTCTCAGGTACTCTGAGGTGACATCACC | |
| | ATTATGTAGGAGGAGGCTATTTCCTGGAGAAGGGGTTCGCTTTCCTTTGACATCTTTTTTCCCTTATGA | |
| | GGACTATCAATTCTTTGGGGGTAGTTCTCCATCTCTGGGGGCCCATTTCCTGGAGGGGATTTTCTGTTT | |
| | CCTGGTGATTGCCCATATCTGGGGGCGTCTTTGGTCCCTTGGGGGAGAGTCTTAGGTTCCTTGAAAGTT | |
| | TCCATGGGGGGCTGTTCTCTGGGTTGGGGGGTCTCATTCTTAAGGGAGTGACCAGCTTTTGGGGGGGGT | |
| | CCCTATTCCCAGAAGAACTTTCCTGGGGGCTTATGCAGCTTCTCCGTTCACTAGAGGACCCCTCCTCTA | |
| | GCATTTCCTGTTTCTGGGAGATGCCTCTTTCTGGATGACCCTTCTCTTTCCAGTGACCCCTTTTGTGGA | |
| | ACCTCCTCCCTTGAGGACCTCCTGATCCTCTGTCCCTCACACAGGTCATGATGTTTGGCAGCACCGCCA | |
| | TCGCCCTGGAGCTGCTGAAGCAAGGTGCCAGCCCCAATGTCCAGGACACCTCCGGTACCAGTCCAGTCC | |
| | ATGACGCAGCCCGCACTGGATTCCTGGACACCCTGAAGGTCCTAGTGGAGCACGGGGCTGATGTCAACG | |
| | TGCCTGATGGCACCGGGGCACTTCCAATCCATCTGGCAGTTCAAGAGGGTCACACTGCTGTGGTCAGCT | |
| | TTCTGGCAGCTGAATCTGATCTCCATCGCAGGGACGCCAGGGGTCTCACACCCTTGGAGCTGGCACTGC | |
| | AGAGAGGGGCTCAGGACCTCGTGGACATCCTGCAGGGCCACATGGTGGCCCCGCTGTGATCTGGGGTCA | |
| | CCCTCTCCAGCAAGAGAACCCCGTGGGGTTATGTATCAGAAGAGAGGGGAAGAAACACTTTCTCTTCTT | |
| | GTTTCTCCTGCCCACTGCTGCAGTAGGGGAGGAGCACAGTTTGTGGCTTATAGGTGTTGGTTTTGGGGG | |
| | TGTGAGTGTTTGGGGGACGTTTCTCATTTGTTTTTCTCACTCCTTTTGGTGTGTTGGACAGAGAAGGGC | |
| | TCCTGCAGGCCACAGCCACCTAAACGGTTCAGTTTCTTCTGCGCCTCAGGCTGCTGGGGCCTCAGACGA | |
| | GACCCAAGGGCAGAGCATTTAAGAGTGAAGTCATGACCTCCAGGGAGCCTAGAAGCTGGTGGCCTTGGC | |
| | CGGCTGTGCTCAGAGACCTGAAGTGTGCACGTTGCTTCAGGCATGGGGGGTGGGGGGAGCGTCCCAAAT | |
| | CAATAAGAAGGTAGAATGAGTTATGAGTTATTCATATTCTGTTGGAAGCTTGTTTTCCAGTCTCTTGTA | |
| | CAGCGTTTTAAAAGAAATGGATTCTATTTATTATGCTTTATTGGAAAAAATGTTGTAATAATTTAATGT | |
| | TTTTACCCATTAAATTAAGACTTGTGCATGATCACA | |

Methods of detection can be in vitro and can comprise obtaining a sample from the subject. Suitable samples may comprise blood, serum, plasma, brain homogenate, interstitial fluid, cerebral spinal fluid, and/or exocrine gland secretion, and enriched forms thereof. According to one embodiment, the sample is a biological sample from a subject in need thereof, such as from a subject that may be suspected of having or at risk for developing an NFT-associated disease, and/or monitoring of the effectiveness of a treatment.

Methods of detection can be in vivo and can comprise, for example, administering a labeled p19INK4D detecting compound to the subject. Other biomarkers can be utilized herein for the identification, monitoring, detection and treatment of NFT-associated disorders, such as nucleic acids, proteins, metabolites and reaction products thereof. Such biomarkers also encompass the mutations, variants, modifications, fragments, and polymorphisms of said biomarkers. In some embodiments, the subject is in need of diagnosis of, or is suspected of having, an NFT-associated disease, and optionally monitoring of the effectiveness of a treatment or progression of a disease. Accordingly, the biomarkers (e.g. indicating CDKN2D and/or p19 expression) are useful in methods of diagnosing, prognosing and/or staging an NFT-associated disease or disorder in a subject by detecting a first level of expression, activity and/or function of one or more biomarkers and comparing the detected level to a control of level wherein a difference in the detected level and the control level indicates a change in expression. Accordingly, in an aspect, a method of monitoring the progress of a neurofibrillary-associated disease in a subject comprises determining a first level of p19INK4D expression in a biological sample obtained from the subject at a first time point; determining a second level of p19INK4D expression in a biological sample obtained from the subject at a second time point; and comparing the second level of p19INK4D expression with the first level of p19INK4D expression; wherein the determining comprises contacting the biological sample with a detection composition; and quantitating the binding to p19INK4D present in the sample. In an aspect, the first time point is a time point before initiation of a treatment regimen, and the second time point is a time point after initiation of a treatment regimen.

The methods may find use in the prediction of a disease and may refer to an advance indication of a disease or condition in a subject not (yet) having the NFT associated disease or condition. The prediction of an NFT-associated disease or condition in a subject may indicate a probability, chance or risk that the subject will develop an NFT-associated disease or condition at a future time. The risk, probability or chance of an NFT-associated disease or condition may be indicated by a range, relative to a control subject, population or baseline, e.g., relative to a general, normal or healthy subject or subject population, and may be indicated as increased or decreased, or as fold-increased or fold-decreased relative to the control or baseline level.

The methods may find use in the prediction of a disease and may refer to an advance indication of a disease or condition in a subject not (yet) having the neuronal senescence associated disease or condition. The prediction of a neuronal senescence associated disease or condition in a subject may indicate a probability, chance or risk that the subject will develop an neuronal senescence associated disease or condition at a future time. The risk, probability or chance of developing a neuronal senescence associated disease or disorder may be indicated by a range, relative to a control subject, population or baseline, e.g., relative to a general, normal or healthy subject or subject population, and may be indicated as increased or decreased, or as fold-increased or fold-decreased relative to the control or baseline level.

CDKN2D, p19INK4D and/or biological markers indicative of the same may be detected or isolated by immunofluorescence, immunohistochemistry (IHC), RNA sequence methods including scRNA seq and snRNA seq, fluorescence activated cell sorting (FACS), mass spectrometry (MS), mass cytometry (CyTOF), time of flight modalities, PCR technologies, including quantitative RT-PCRs, FISH technologies, in situ hybridization, etc. Absorbance assays, colorimetric assays can be utilized dependent on the detectable labels and approaches desired. In certain applications, the detection may comprise primers and/or probes or fluorescently bar-coded oligonucleotide probes for hybridization to RNA (see e.g., Garg et al., Cancers (Basel). 2022 August; 14(15): 3628; doi: 10.3390/cancers14153628, incorporated herein in its entirety for its teachings of sample processing an analytical imaging and techniques).

Detectable labels can be utilized in the compositions of the present invention. In an aspect, the labels are designed to be specific for p19. In vivo imaging can include CT, MRI, and PET, with molecular imaging using radioactive and/or optical probes. See, Freise and Wu, Mol. Immunol. 2015 October; 67(2 0 0): 142-152; doi: 10.1016/j.molimm.2015.04.001; Krishnaswamy et al., J Neurosci. 2014 Dec. 10; 34(50): 16835-16850; doi: 10.1523/JNEUROSCI.2755-14.2014. In an example embodiment, the label is a radioligand for PET imaging and may comprise [¹¹C] or [¹⁸F]. Methods for labeling compounds useful to detect biomarkers are well known. See, e.g. U.S. Pat. No. 10,865,207 discussing methods of labeling with fluorinating agents, including H¹⁸F, alkali or alkaline earth ¹⁸F-fluorides or tetraalkyl ammonium salt or tetraalkyl phosphonium salts of ¹⁸F. See also, L. Cai, S. Lu, V. Pike, Eur. J. Org. Chem 2008, 2853-2873; J. Fluorine Chem., 27 (1985): 177-191; Coenen, Fluorine-18 Labeling Methods: Features and Possibilities of Basic Reactions, (2006), in: Schubiger P. A., Friebe M., Lehmann L., (eds), PET-Chemistry—The Driving Force in Molecular Imaging. Springer, Berlin Heidelberg, pp. 15-50.

The disease to be treated that is associated with NFT in a subject and/or associated with increased CDKN2D gene expression in a subject, as taught herein, may be any disease, disorder or condition now known or later identified to be associated with increased CDKN2D gene expression in NFT in a subject. In some embodiments, the disease, disorder, or condition is a neurodegenerative disease. The neurodegenerative disease to be treated may include, but is not limited to, amyotrophic lateral sclerosis (ALS), frontotemporal lobar degeneration (FTLD), Huntington's disease, Parkinson's disease, Alzheimer's disease, etc. In some embodiments, the disease to be treated is associated with the presence of NFTs in a subject or associated with increased cellular senescence and increased CDKN2D gene expression or presence of p19INK4D protein in a subject.

The disease or disorder associated with cellular senescence may include, but is not limited to, Aicardi Goutiere's syndrome, progressive supra nuclear palsy (PSP), osteoarthritis, cardiovascular dysfunction, atherosclerosis. Osteoporosis, chemotherapy-induced adverse effects such as blood clots, bone marrow suppression, cardiotoxicity, cardiovascular Alzheimer's disease, amyotrophic lateral sclerosis (ALS), Huntington's disease, vision loss, hearing loss, peripheral degenerative diseases, frontotemporal dementia (FTD), multiple sclerosis (MS), Aicardi Goutiere's syndrome, progressive supra nuclear palsy (PSP), hematopoietic stem cell function, Parkinson's disease, pulmonary fibrosis, wound healing, and/or tissue regeneration. See, e.g. Lopez-Otin, C., et al. (2013). The hallmarks of aging. Cell 153, 1194-1217; Demaria, M., et al. (2017). Cellular Senescence Promotes Adverse Effects of Chemotherapy and Cancer Relapse. Cancer Discov 7, 165-176; Demaria, M., et al. (2014). Dev Cell 31, 722-733; Franceschi, C. & Campisi, J. (2014). J. Gerontol. A Biol. Sci. Med. Sci. 69 Suppl. 1, S4-9.

The treatment methods disclosed herein may comprise treatment that results in reduction in the underlying pathology, e.g. reduction in NFTs. In another embodiment, the underlying pathology remains about the same, and there is not any evidence of progression. In some embodiments, any change in the underlying pathology may be identified by detection of a biomarker before and after the administration. In one embodiment, the biomarker is detected by PET imaging. In another embodiment, the underlying pathology is identified by measurement of the amount of NFT before and after the treatment.

The methods taught herein may serve to improve a range of physical, mental, and emotional attributes of the treated subject. In one embodiment, symptoms of mild cognitive impairment and any change in the symptoms of Alzheimer's disease is determined using the criteria set forth in DSM-5. The subject can show an improvement in one or more symptoms of a neurodegenerative disease. Such improvements include, but are not limited to, improved physical abilities such as fine motor skills (e.g., writing and typing, grasping small objects, cutting, pointing, etc.), or gross motor skills (e.g., walking, balance, jumping, standing up, throwing); improved sensations such as decreased tingling and/or increased sensitivity in extremities, reduced sensation of muscle weakness or rigidity, and reduced tremors or pain; improved cognitive abilities such as increased alertness, reduced memory loss/improved memory recall, increased cognitive comprehension, improved speech and sleep, improved puzzle-solving abilities, increased focus; and improved behavioral performance such as decreased apathy, depression, agitation, or anxiety, and improved mood and general contentment.

In some embodiments, the methods treat or prevent a disease, disorder, or condition by reducing the rate of NFT formation in the subject (e.g., reducing the rate of formation of protein inclusions). In some embodiments, the methods treat a disease, disorder, or condition by reducing the amount NFT in the subject (e.g., reducing the amount of protein inclusions). In some embodiments, the methods prevent NFT formation in the subject. Thus, the methods can reduce and/or prevent formation of pathological inclusions in cells of a subject. Some embodiments of the present disclosure provide a composition for inhibiting cellular senescence caused due to NFT formation, accumulation and/or aggregation.

In some embodiments, the methods can disaggregate existing protein aggregates. Thus, the methods can reduce the amount of existing protein aggregates prior to beginning the methods. This can be useful for patients experiencing neurodegenerative disease symptoms, as such patients are likely to have existing protein aggregates. Disaggregation of existing aggregates can be, but need not necessarily be, in addition to prevention or reduction of further aggregate formation. As used herein, the term “disaggregate” refers to the breaking down of one or more protein aggregates, for example in an NFT. As a protein aggregate contains numerous copies of a protein clumped together, “disaggregation” refers to a process of removing portions of the aggregated protein clump. Thus, as used herein, “disaggregation” refers to the removal of portions of an existing protein aggregate, such that after disaggregation, the result is a smaller protein aggregate clump or an absence of a protein aggregate clump altogether. Detection of aggregate size and changes thereto depend on the sensitivity of the equipment and techniques used to detect aggregate size. Thus, under one technique, a disaggregated clump or tangle may be undetectable, whereas under another technique, the same disaggregated clump or tangle may be detected as having a smaller size.

The methods may generate neuroprotective results when performed in a subject. As used herein, the term “neuroprotective” refers to maintaining or improving existing neurological function in the target neurological organ or tissue (e.g., nerve, spinal cord), or can refer to maintaining or improving the rate or overall amount of neuronal cell death in target neuronal cells. For example, “neuroprotective” can refer to slowing the rate of nerve tissue destruction, deterioration, or malfunction, slowing the rate of neuronal cell death, reducing the rate at which nerve conduction speed slows, etc. In some embodiments, the methods can generate at least 5%, at least 10%, at least 20%, or at least 25% or more neuroprotective improvement, as compared to a control.

### Therapeutics

Therapeutics that can be used for treatment of a the NFT-associated or cellular senescence disease or disorder can comprise modulating agents, e.g., genetic modifying agents, antibodies or antigen binding fragment thereof, or small molecules. In a preferred embodiment, the modulating agents specifically bind the CDKN2D or p19INK4D or a fragment thereof to thereby inhibit expression and/or activity. Additional therapeutics can include treatment modalities administered upon a finding of the presence of NFT or increased risk of the presence of NFT associated disorders in a subject according to the methods of detection. Additional therapeutics can include treatment modalities administered upon a finding of the cellular senescence associated disorders or increased risk of the presence of cellular senescence associated disorders in a subject according to the methods of detection. In addition to directly targeting CDKN2D and/or the resulting protein, the treatments can include one or more cognitive enhancers such as Memantine, Rivastigmine, Galantamine and Donepezil, senolytics (dasatinib with quercetin, fisetin, etc) among others, for concurrent administration.

### Antibodies

Antibodies specific to the p19INK4D protein can be utilized in the methods of the invention. In some embodiments, the antibody, or antigen binding fragment thereof is specific for the p19INK4D protein. Polyclonal or monoclonal antibodies may be raised against the p19INK4D protein, for example. Commercially available antibodies, for example, monoclonal Anti-p19INK4d antibody produced in mouse from Sigma-Aldrich® can be utilized. The term “antibody fragment” refers to a portion of an immunoglobulin, often the hypervariable region and portions of the surrounding heavy and light chains that displays specific binding affinity for a particular target, typically a molecule. A hypervariable region is a portion of an immunoglobulin that physically binds to the polypeptide target. An antibody fragment thus includes or consists of one or more portions of a full-length immunoglobulin retaining the targeting specificity of the immunoglobulin. Such antibody fragment may for instance lack at least partially the constant region (Fc region) of the full-length immunoglobulin. In some embodiments, an antibody fragment is produced by digestion of the full-length immunoglobulin. An antibody fragment may also be a synthetic or recombinant construct that contains one or more parts of the immunoglobulin or immunoglobulin chains (see e.g. HOLLIGER, P. and Hudson, J. Engineered antibody fragments and the rise of single domains. Nature Biotechnology 2005, vol. 23, no. 9, p. 1126-1136). Examples of an antibody fragment include, but are not limited to, an scFv, a Fab, a Fv, a Fab′, a F(ab′)2 fragment, a dAb, a VHH, a nanobody, a V(NAR) or a so called minimal recognition unit.

A “single chain variable fragment” or a “single chain antibody” or an “scFv” are examples of a type of antibody fragment. An scFv is a fusion protein that includes the VH and VL domains of an immunoglobulin connected by a linker. It thus lacks the constant Fc region present in a full-length immunoglobulin.

A “monoclonal antibody or an antigen binding fragment thereof” as used herein refers to a full-length immunoglobulin, an antibody fragment, a proteinaceous non-immunoglobulin scaffold, and/or other binding compound, which has an immunoglobulin-like function. Typically the monoclonal antibody or an antigen binding fragment thereof is a proteinaceous binding molecule. Such monoclonal antibody or an antigen binding fragment thereof can be monovalent or multivalent, i.e. having one or more antigen binding sites. Non-limiting examples of monovalent binding members include scFv, Fab fragments, dAb, VHH, DARPins, affilins and nanobodies. A multivalent monoclonal antibody or an antigen binding fragment thereof can have two, three, four or more antigen binding sites whereby one or more different antigens can be recognized. Full length immunoglobulins, F(ab′)₂ fragments, bis-scFv (or tandem scFv) and diabodies are nonlimiting examples of multivalent monoclonal antibody or an antigen binding fragment thereof; in the exemplary multivalent monoclonal antibody or an antigen binding fragment thereof, two binding sites are present, i.e. the monoclonal antibody or an antigen binding fragment thereof is bivalent. In some embodiments, the multivalent monoclonal antibody or an antigen binding fragment thereof is bispecific, i.e. the monoclonal antibody or an antigen binding fragment thereof is directed against two different targets or two different target sites on one target molecule. Bispecific antibodies are, e.g., reviewed in MULLER, D. and Kontermann, R. E. Bispecific antibodies. Edited by DUBEL, S. Weinheim: Wiley-VCH, 2007. ISBN 3527314539. p. 345-378. In some embodiments, the multivalent monoclonal antibody or an antigen binding fragment thereof includes more than two, e.g., three or four different binding sites for three or four, respectively, different antigens. Such monoclonal antibody or an antigen binding fragment thereof is multivalent and multispecific, in particular tn- or tetra-specific, respectively.

“Non-antibody scaffolds” are antigen-binding polypeptides which are e.g. described in FIELDER, M. and Skerra, A. Non-antibody scaffolds. Edited by DUBEL, S. Weinheim: Wiley-VCH, 2007. ISBN 3527314539. p. 467-500; or GILBRETH, R. N. and Koide, S. Structural insights for engineering binding proteins based on non-antibody scaffolds. Curr Opin Struct Biol 2012, vol. 22, p. 4 13-420. Non-limiting examples include affibodies, affilin molecules, an AdNectin, a mutein based on a polypeptide of the lipocalin family (Anticalin®), a DARPin, Knottin, a Kunitz-type domain, an Avimer, a Tetranectin and a trans-body. Avimers contain so called A-domains that occur as strings of multiple domains in several cell surface receptors (Silverman, J., et al., Nature Biotechnology (2005) 23, 1556-1561). Tetranectins, derived from the respective human homotrimeric protein, likewise contain ioop regions in a C-type lectin domain that can be engineered for desired binding (ibid.).

### Genetic Modifying Agents

In an example embodiment, a method of treatment comprises administering a RNAi therapeutic to reduce expression of CDKN2D and/or the protein it encodes, p19. A RNAi therapeutic comprises a polynucleotide that is complementary to a portion of the target sequence mRNA, generally ranging in size from 15 to 50 base pairs. In an example embodiment, the siRNA is a nucleic acid that can form a double stranded RNA with the ability to reduce or inhibit expression of a gene or target gene. Each complementary sequence of the double stranded siRNA is about 15-50 nucleotides in length, and the double stranded siRNA is about 15-50 base pairs in length. A small hairpin RNA (shRNA) is also contemplated for use. The shRNA is an antisense strand of about 19 to about 25 nucleotides followed by a short nucleotide loop (approximately 5 to 9 nt) followed by the analogous sense strand. In an embodiment, an RNAi is a microRNA or miRNA, endogenous RNAs, some of which are known to regulate the expression of protein-coding genes at the posttranscriptional level. See, e.g., Lim et al Science 299, 1540 (2003), Lee and Ambros Science, 294, 862 (2001), Lau et al., Science 294, 858-861 (2001), Lagos-Quintana et al, RNA, 9, 175-179 (2003).

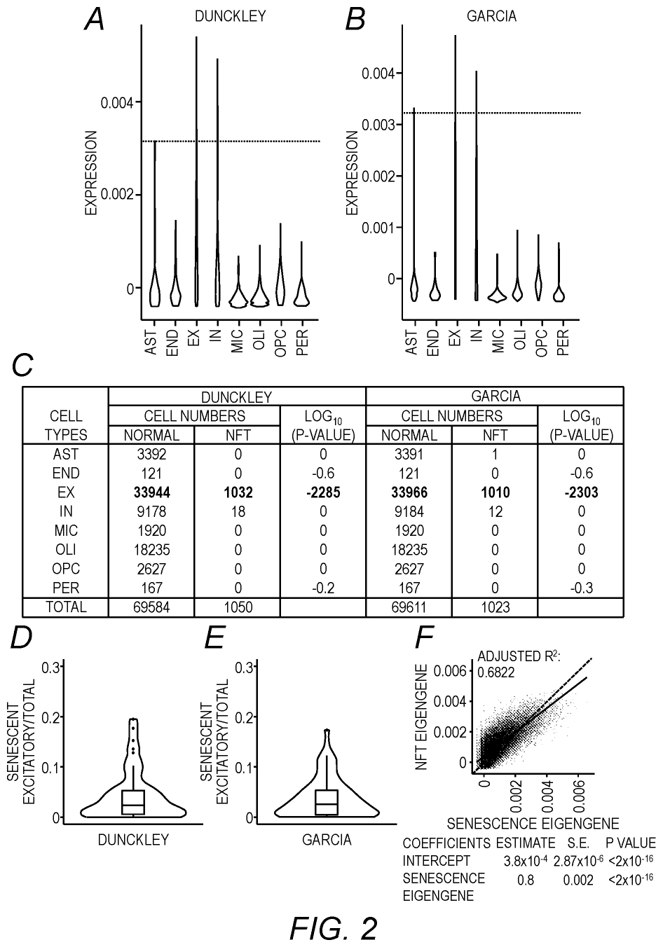

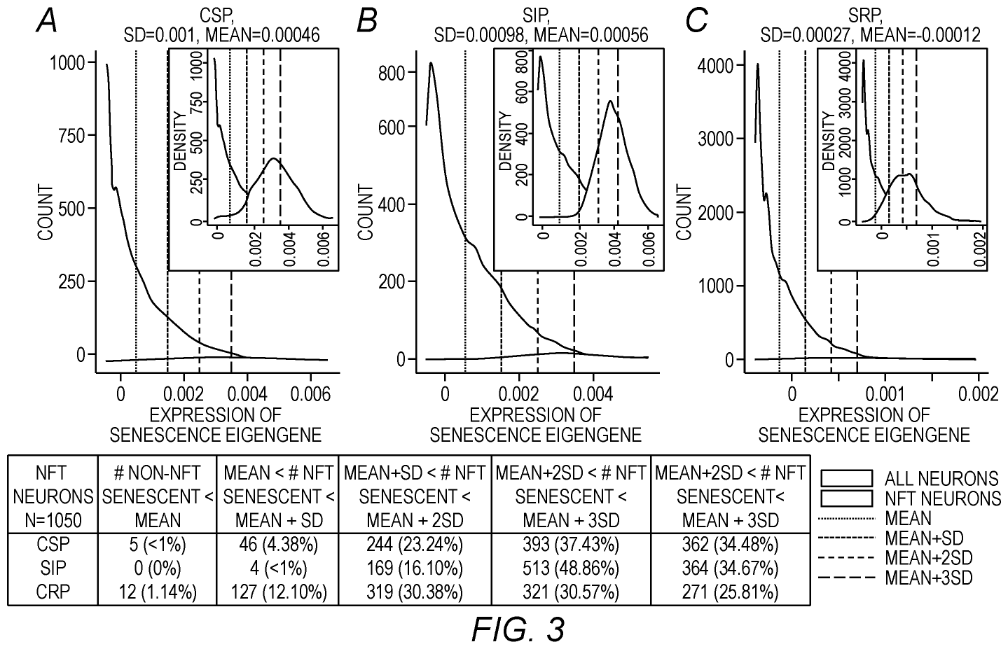

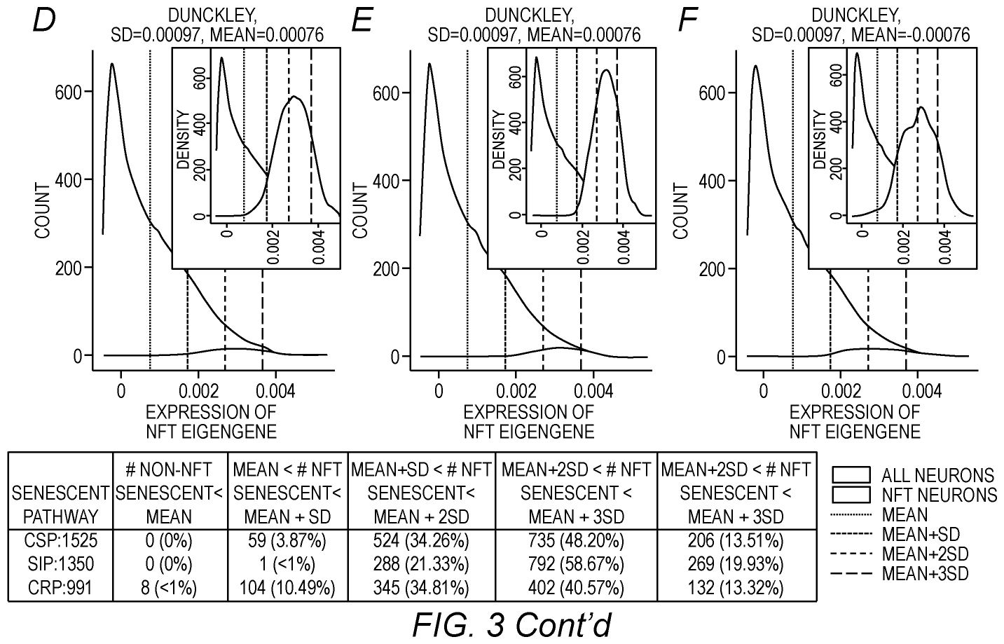

Different criteria are available for selecting the nucleic acid for use and may comprise scanning the mRNA sequence of the target, and may include empiric determination in accordance with, for example, Sui G et al., Proc. Natl. Acad. Sci. USA 99:5515-20 (2002), and may include confirmation the sequence lacks significant sequence homology with other genes as analyzed by BLAST search. Additional approaches may comprise any accessible site in endogenous mRNA can be targeted for degradation by synthetic oligodeoxyribonucleotide/RNase H method (see, e.g., Lee N S et al., Nature Biotechnol. 20:500-05 (2002)). RNAi treatment may comprise miRNA or siRNA. or a pre-miRNA which is processed by Dicer to form a miRNA. The RNAi may also comprise a dsRNA or shRNA which is processed by Dicer to form a siRNA. The polynucleotides may comprise one or more modifications to suppress innate immune activation, enhance activity and specificity, and reduce off-target induced toxicity. Example teachings can be found, for example at Provost et al., E.M.B.O. J., 2002 Nov. 1; 21(21): 5864-5874; Tabara et al., Cell 2002 Jun. 28; 109(7):861-71; Martinez et al., Cell 2002 September. 6; 110(5):563; Hutvagner & Zamore, Science 2002, 297:2056. In certain embodiments, a single-stranded RNAi agent disclosed herein can comprise substitutions, or modifications, including chemically modified nucleotides, and non-nucleotides which may include incorporation in the backbone, sugars, bases, or nucleosides. The use of substituted or modified single-stranded RNAi agents can be designed to have an increased half-life in a subject. Furthermore, certain substitutions or modifications can be used to improve the bioavailability of single-stranded RNAi agents by targeting particular cells or tissues or improving cellular uptake of the single-stranded RNAi agents. Exemplary modifications and locations within a RNAi polynucleotide are described in Hu et al. “Therapeutic siRNA: state of the art” Signal Transduction and Targeted Therapy 5, Article number 100 (2020), incorporated herein by reference, see, e.g. FIGS. 2 and 3, specifically for its teachings of modifications.

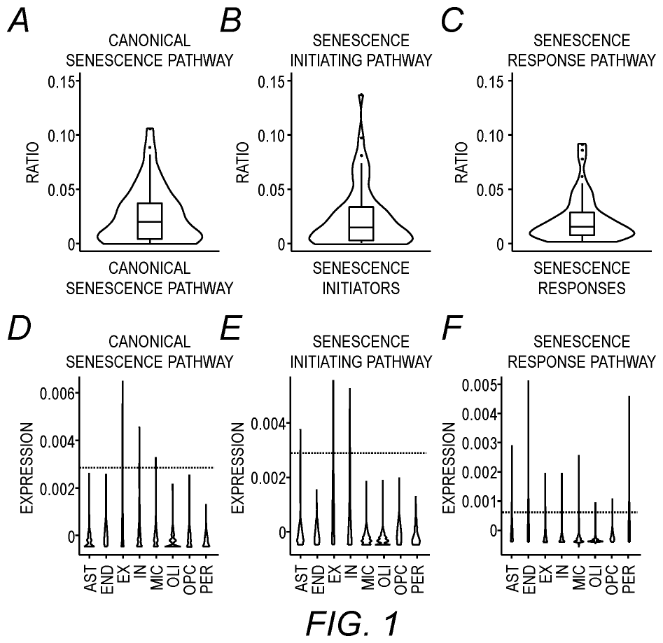

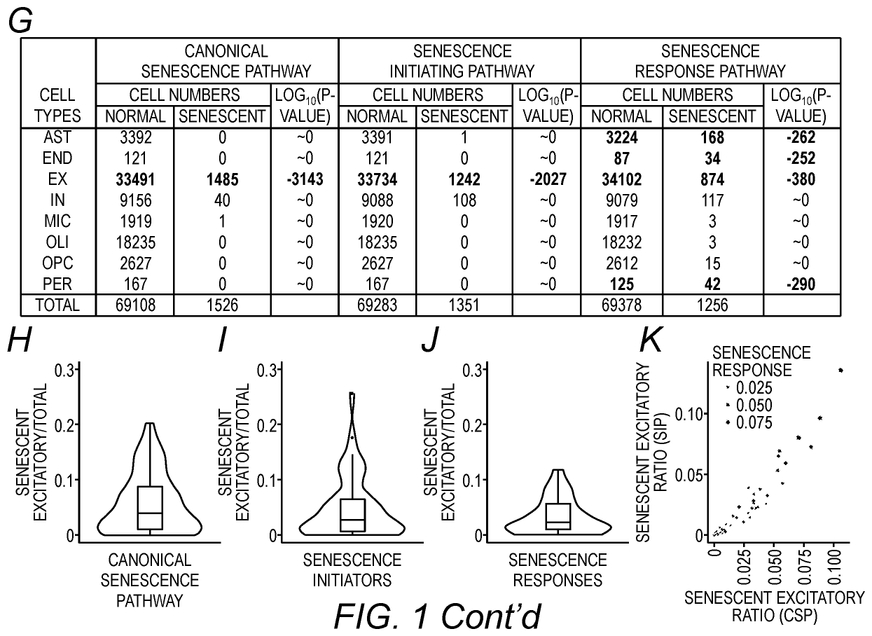

Gene editing systems can also be utilized, which may comprise a CRISPR system, a zinc finger nuclease system, or a TALE system. A CRISPR-Cas system can comprise a Class 1 or Class 2 CRISPR-Cas system, which may comprise a guide sequence engineered to specifically bind a polynucleotide of interest. The CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18:67-83, particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2: Type V systems include V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems include VI-A, VI-B1, VI-B2, VI-C, and VI-D. Design of guides for targeting a nucleic acid for modification is known in the art, see, e.g. IDTdna.com and Synthego.com for guidance on custom guide RNAs. Reduction of off-target effects can be tailored using programs such as GUIDE-seq. See, e.g. Malinin, et al., Nature Protocols, 16, 5592-5615 (2012). TALEN based gene editing is also contemplated and can be used in in vivo applications. See, S Becker, J Boch—Gene and Genome Editing, 2021. Zinc finger nuclease editing can also be utilized, and further modified to ensure high-precision gene editing. See, e.g., Conway et al., Molecular Therapy, 27:4, 10 Apr. 2019, Pages 866-877; Paschon et al. Nature Comm, 10:1133 (2019). Gene editing tools are well known in the art, with advantages and comparison of the tools that can be considered for the desired application. Rahim et al., Int'l J. of Innovative Science and Research Tech., 6:8 (2021), incorporated herein by reference.

Embodiments of the present disclosure further include methods comprising using a single-stranded RNAi agent, and methods for inhibiting expression of one or more corresponding target mRNAs in a cell or organism are thus encompassed according to the invention. Example delivery vectors of RNAi including viruses are described in Nguyen et al. “RNAi therapeutics: An update on delivery” (2008). Current Opinion in Molecular Therapeutics 10(2): 158-167; and Lundstrom, “Viral Vectors Applied for RNAi-Based Antiviral Therapy” Viruses (2020) 12, 924 doi:10:3390/v12092924.

A substantial change in the expression level of mRNA or of the protein encoded by the CDKN2D gene after the introduction of the RNAi, e.g. siRNA sequence, is indicative of the effectiveness of the siRNA sequence in suppressing the expression of the target gene. In one specific example, the expression levels of other genes are also monitored before and after the introduction of the siRNA sequence. An siRNA sequence which has inhibitory effect on target gene expression but does not significantly affect the expression of other genes can be selected.

Treatments can be tailored for a variety of NFT-associated and cellular senescence-associated diseases which are known in the art. By way of example, the discussion below is directed to treatments that may be used for Alzheimer's Disease upon detection of increased CDKN2D gene expression and/or p19INK4D protein expression.

In an embodiment, treatment may comprise use of bioactive natural compounds, including phenolic compounds, omega-3 fatty acids, fat-soluble vitamins, isothiocyanates, and carotenoids via supplements or nutrition. See, e.g. Grodzicki, Dziendzikowska. 2020. “The Role of Selected d Bioactive Compounds in the Prevention of Alzheimer's Disease” Antioxidants 9, no. 3: 229doi:10.3390/antiox9030229. In one embodiment, treatment may comprise increasing physical activity, which may have potential to delay disease progression in presymptomatic subjects. See, e.g. De la Rosa et al, J. of Sport and Health Sci. 9 (2020) 394-404.

Treatment can comprise administration of Aducanumab. Aducanumab is an FDA-approved therapy for treatment of Alzheimer's disease. In an aspect, the aducanumab is administered intravenously (IV) via a 45- to 60-minute infusion every 4 weeks.

Additional treatment may include management of symptoms, including use of cholinesterase inhibitors and memantine. Example cholestinerase inhibitors can comprise Donepezil (Aricept) for use at an early stage, and taken once daily as a pill, Galantamine (Razadyne) for mild to moderate Alzheimer's administered as a pill once daily or as an extended-release capsule twice a day; and Rivastigmine (Exelon) is approved for mild to moderate Alzheimer's disease which may be administered as a pill or used as a patch to treat severe Alzheimer's disease. Another approved treatment is the combination of donepezil and memantine (Namzaric), which is taken as a capsule.

Four monoclonal antibodies anti-tau (Gosuranemab, Tilavonemab, Semorinemab and Zagotenemab) and one anti-tau vaccine (AADvac1) are currently under clinical trials, and may be used as a therapeutic. Additionally, nerve growth factor (NGF), brain derived neutrotrophic factor (BDNF), APP, PSEN1 and PSEN2, and APOE modulating has been contemplated for potential treatment. See, Thoe et al., Life Sciences 276 (2021) 119129, incorporated herein by reference at pages 6-7 and Table 1. Amyloid beta immunotherapy through vaccination is also contemplated as treatment, including vaccinations in clinical testing. See, id at Table 3. Passive immunotherapeutic studies are also contemplated, including the aforementioned aducanumab as well as other antibody-based treatments. See Thoe, at Table 4, incorporated herein by reference.

Delivery of therapeutics via liposomes and nanoparticles are provided. In an aspect, the systems may comprise conjugation to a desired treatment including, for example, curcumin, antioxidants, antibodies and the other therapeutic compositions detailed elsewhere herein. In embodiments, metal nanoparticles, such as gold nanoparticles (AuNPs), silver nanoparticles (AgNPs) and metal oxide nanoparticles, which may be delivered alone or coupled to an additional moiety for treatment can be utilized in treatment and have been shown to circumvent the blood brain barrier.

The delivery vehicle may comprise a nanoparticle comprising modified dendrimers for the enclosure of the delivery of a nucleic acid, e.g. RNAi therapeutic. Exemplary dendrimers include polyester dendrimers, which may be modified with amin linkers, fatty acid derivatives, etc. Exemplary molecules and method of making may be found at International Publication WO 2020/132196. Lipid particles, for example, lipid nanoparticles and liposomes, may also be used. In an example embodiment, the lipid particles comprise one or more CDKN2D polynucleotides encoding a p19INK4D polypeptide according to the present invention. Example lipid nanoparticles can be found in the art, for example, in U.S. Pat. Nos. 9,868,692, 10,266,485, 10,442,756, and 10,272,150. Liposomes and stable nucleic acid lipid particles (SNALPs) can also be used for delivery.

Administration of particular therapeutics may comprise determining an Alzheimer's stage, mild, moderate or severe, which may be determined based on assessment of memory, awareness of time and place, thinking and reasoning, and/or imaging modalities such as the use of compositions of the present invention for the detection of NFTs. The treatment may be utilized in subjects with mild cognitive impairment (MCI) or who are otherwise at risk of the development of Alzheimer's Disease.

The therapeutics described herein can be utilized in methods for modulating the presence of neurofibrillary tangles in a cell, comprising: administering to the subject a therapeutically effective amount of a composition that specifically inhibits the activity of CDKN2D or p19INK4D, wherein the administration inhibits the formation and/or presence of neurofibrillary tangles in the subject. The modulating the presence of neurofibrillary tangles in a cell comprises preventing the formation of, or reducing the presence of, neurofibrillary tangles in the cell. In a preferred embodiment, the cell is a neuron, preferably an excitatory neuron.

The therapeutics described herein can be utilized in methods for inhibiting cellular senescence caused by or associated with neurofibrillary tangles in a subject, comprising: administering to the subject a therapeutically effective amount of a composition that specifically inhibits CDKN2D or p19INK4D.

The therapeutics described herein can be utilized in methods of disaggregating NFTs in a subject, the method comprising administering to the subject one or more modulating agents of CDK2ND, p19INK4D or functional fragments thereof, nucleic acid molecules, or other therapeutics of the invention, thereby disaggregating NFTs in the subject.

The therapeutics described herein can be utilized in methods of inhibiting formation of NFTs comprising CDKN2D or p19INK4D in a subject, the method comprising administering to the subject any one of the modulating agents or functional fragments thereof, nucleic acid molecules, or other therapeutics of the invention, thereby inhibiting formation of NFTs comprising CDKN2D or p19INK4D in the subject.

The administering step of any one of the methods described herein can include at least one, at least two, at least three, at least four, at least five, at least six, at least seven, at least eight, at least nine, or at least ten dosages. The administering step can be performed before the subject exhibits disease symptoms (e.g., prophylactically), or during or after disease symptoms occur. The administering step can be performed prior to, concurrent with, or subsequent to administration of other agents to the subject. In some embodiments, the administering step is performed prior to, concurrent with, or subsequent to the administration of one or more additional diagnostic or therapeutic agents.

### Pharmaceutical Compositions

As a further aspect, the invention provides pharmaceutical formulations and methods of administering the same to achieve any of the therapeutic effects (e.g., treatment of tauopathy) discussed above. The pharmaceutical formulation may comprise any of the therapeutics discussed above in a pharmaceutically acceptable carrier. By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like.

One embodiment of the invention is a composition including an isolated polynucleotide sequence capable of modulating a CDKND molecule encoding a p19INK4D or functional fragment thereof, a plasmid or vector containing the isolated polynucleotide sequence, or a transfected cell containing the plasmid or vector or the isolated polynucleotide sequence and a suitable carrier, diluent, or excipient, and optionally a pharmaceutically acceptable carrier, diluent, or excipient.

Therapeutics, for example, modulating agents, can be formulated for administration in a pharmaceutical carrier in accordance with known techniques. See, e.g., Remington, The Science And Practice of Pharmacy (23rd Ed. 2020). In the manufacture of a pharmaceutical formulation according to the invention, the p19INK4D or CDKN2D modulating agent (including the physiologically acceptable salts thereof) is typically admixed with, inter alia, an acceptable carrier. The carrier can be a solid or a liquid, or both, and is preferably formulated \ as a unit-dose formulation, for example, a tablet, which can contain from 0.01 or 0.5% to 95% or 99% by weight of the modulating agent. One or more modulating agents can be incorporated in the formulations of the invention, which can be prepared by any of the well-known techniques of pharmacy.

Suitable carriers include, but are not limited to, salts, diluents. (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, anti-infective agents, suspending agents, wetting agents, viscosity modifiers, tonicity agents, stabilizing agents, and other components and combinations thereof.

Suitable pharmaceutical carriers and their formulations are described in Remington's Pharmaceutical Sciences, 23rd ed. 2020, Academic Press. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions. Typically, the components of the formulation are dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol. In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers. In some embodiments, the formulation can be distributed or packaged in a liquid form, or alternatively, as a solid, obtained, for example by lyophilization of a suitable liquid formulation, which can be reconstituted with an appropriate carrier or diluent prior to administration.

A further aspect of the invention is a method of treating subjects, comprising administering to a subject a pharmaceutical composition comprising a modulating agent, e.g. a composition capable of specifically binding p19INK4D or CDKN2D polynucleotides in a pharmaceutically acceptable carrier, wherein the pharmaceutical composition is administered in a therapeutically effective amount. Administration of the composition according to the present invention to a human subject or an animal in need thereof can be by any means known in the art for administering compounds.

The most suitable route in any given case will depend on the nature and severity of the condition being treated and on the nature of the particular compound which is being used. In an embodiment, administration may be direct delivery to the cerebrospinal fluid (CSF) via intrathecal delivery, or administration utilizing delivery systems that can cross the blood brain barriers, e.g. via AAV vectors such as AAV9. In some embodiments, it may be desirable to deliver the formulation locally to avoid any side effects associated with systemic administration. For example, local administration can be accomplished by direct injection at the desired treatment site, such as the brain areas in which CDKN2D expression is detected. In some embodiments, the formulation can be a slow-release formulation, e.g., in the form of a slow-release depot.

Further, the present invention provides liposomal formulations of the compounds disclosed herein and salts thereof. The technology for forming liposomal suspensions is well known in the art. When the compound or salt thereof is an aqueous-soluble salt, using conventional liposome technology, the same can be incorporated into lipid vesicles. In such an instance, due to the water solubility of the compound or salt, the compound or salt will be substantially entrained within the hydrophilic center or core of the liposomes. The lipid layer employed can be of any conventional composition and can either contain cholesterol or can be cholesterol-free. When the compound or salt of interest is water-insoluble, again employing conventional liposome formation technology, the salt can be substantially entrained within the hydrophobic lipid bilayer which forms the structure of the liposome. In either instance, the liposomes which are produced can be reduced in size, as through the use of standard sonication and homogenization techniques.

The liposomal formulations can be lyophilized to produce a lyophilizate which can be reconstituted with a pharmaceutically acceptable carrier, such as water, to regenerate a liposomal suspension.

The amount of the therapeutic administered to a subject will vary from subject to subject, depending on the nature of the disclosed compositions and/or formulations, the species, gender, age, weight and general condition of the subject, the mode of administration, and the like. Effective dosages and schedules for administering may be determined empirically, and making such determinations is within the skill in the art. The dosage ranges for the administration of the therapeutic are those large enough to produce the desired effect (e.g., to reduce protein inclusions or to improve a symptom of a neurodegenerative disease). The dosage should not be so large as to outweigh benefits by causing extensive or severe adverse side effects, such as unwanted cross-reactions, anaphylactic reactions, and the like, although some adverse side effects may be expected. The dosage can be adjusted by the individual clinician in the event of any counterindications. Therapeutic administration can include use of the labeled detection compositions detailed herein to determine effective amounts of therapeutics and/or effectiveness of therapy treatments.

Generally, the therapeutics and/or formulations are administered to the subject at a dosage of active component(s) ranging from 0.1 mg/kg body weight to 100 g/kg body weight. In some embodiments, the therapeutics and/or formulations are administered to the subject at a dosage of active component(s) ranging from 1 mg/kg to 10 g/kg, from 10 mg/kg to 1 g/kg, from 10 mg/kg to 500 mg/kg, from 10 mg/kg to 100 mg/kg, from 10 mg/kg to 10 mg/kg, from 10 mg/kg to 1 mg/kg, from 10 mg/kg to 500 mg/kg, or from 10 mg/kg to 100 mg/kg body weight. Dosages above or below the range cited above may be administered to the individual subject if desired.

A further aspect of the invention relates to kits for use in the methods of the invention. The kit can comprise a detection composition of the invention in a form suitable for administration to a subject or sample or in a form suitable for compounding into a formulation. The kit can further comprise other therapeutic agents, carriers, buffers, containers, devices for administration, and the like, including modulating agents as detailed elsewhere herein. The kit can further comprise labels and/or instructions, for treatment of a disorder. Such labeling and/or instructions can include, for example, information concerning the amount, frequency and method of administration of a detection composition or therapeutic composition of the invention.

The kit can comprise the antibody, or antigen binding fragment thereof, of the invention in a form suitable for diagnostic use, or suitable for compounding into a diagnostic or detection formulation. The detection compositions can be formulated for administration to a subject or in vitro use with a biological sample. The kit can further comprise other labeling agents, solid supports, carriers, buffers, containers, devices for administration, and the like. The kit can further comprise labels and/or instructions, for detection of a disorder. Such labeling and/or instructions can include, for example, information concerning measurement amount, background corrections, and method of administration of the antibody.

The kit can comprise an inhibitor of CDKN2D or p19INK4D protein, or a fragment thereof, of the invention in a form suitable for diagnostic use, or suitable for compounding into a diagnostic or detection formulation, or for a therapeutic use. The detection compositions can be formulated for administration to a subject or in vitro use with a biological sample or in vivo administration for detection and treatment, e.g. with a p19INK4D radioligand. The kit can further comprise other labeling agents, solid supports, carriers, buffers, containers, devices for administration, and the like. The kit can further comprise labels and/or instructions, for detection of a disorder. Such labeling and/or instructions can include, for example, information concerning measurement amount, background corrections, and method of administration.

Having described the present invention, the same will be explained in greater detail in the following examples, which are included herein for illustration purposes only, and which are not intended to be limiting to the invention.

### EXAMPLES

### Example 1: Senescent Cells Identified and Quantified in Human Brain

RNA sequencing (sn-RNA-Seq) data generated from two independent studies, Mathys et. al, 2019²² and Zhou et. al, 2020²³, was analyzed and is referred to as cohort 1 and cohort 2, respectively. Datasets were accessed through Accelerating Medicines Partnership-AD (AMP-AD²⁴) with Synapse IDs syn18485175 and syn21126462, respectively. The data included ˜80,000²² and ˜70,000²³ single nuclei derived from 48 and 32 postmortem human brain samples, respectively, from longitudinal cohort studies of aging and dementia: the Religious Order Study (ROS) and the Rush Memory and Aging Project (MAP)²⁵. Four subjects (10248033, 20207013, 10290265, and 11072071) were represented in both datasets. They were included only in cohort 1 but not cohort 2; in total ˜140,000 cells were analyzed from 76 brains.

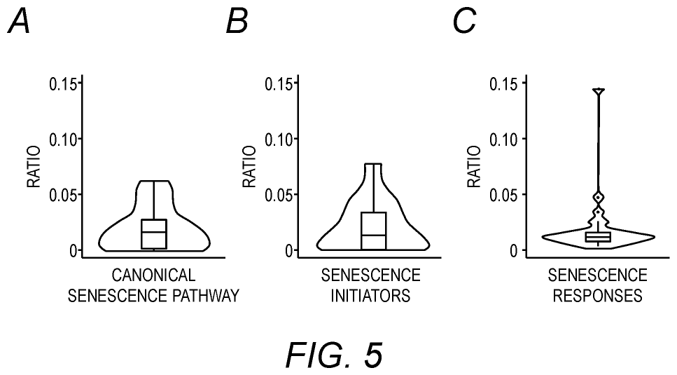

To identify senescent cells, three gene sets representative of cellular senescence that were guided by preclinical studies including those from aged and transgenic mouse models of AD pathology were used⁷⁻¹³. The gene sets reflected 1) a canonical senescence phenotype (CSP) with 22 genes including CDKN2A and CDKN1A that are upregulated in many senescent cell types; 2) 48 genes upregulated early in senescence, which were termed senescence initiating pathway (SIP); and 44 genes upregulated after the stable arrest and involved in a pro-inflammatory secretory phenotype, which were termed senescence response pathway (SRP). For each of these three gene sets, a principal component analysis was preformed to compute an eigengene²⁶, a weighted average expression over all genes in the corresponding list²⁷. Weights were optimized using methods whereby explained variance was maximized, and thus, the loss of biological information is expected to be minimal. The mean expression of each eigengene was computed over all analyzed cells and considered a cell to be senescent if the level of expression of the eigengene was more than the mean expression over all cells plus three times the standard deviation (mean+3sd). Analyses using the CSP eigengene revealed 1526 total senescent cells in the dorsolateral prefrontal cortex (2.1%) from cohort 1; the proportion differed across individuals (0-13%). A total of 1242 cells and 1118 cells expressed the SIP and SRP, respectively (2% and 1.7%), FIG. 1a-c. Similar results were derived from the cohort 2 whereby 1331 (2.3%), 1485 (2.56%) and 951 (1.64%) cells expressed the CSP, SIP and SRP eigengenes, respectively (FIG. 5 and Table 1a-b). Collectively these data indicate that senescent cells comprise ˜2% of the brain cell population in these two independent cohorts.

### Senescent Cell Types Identified

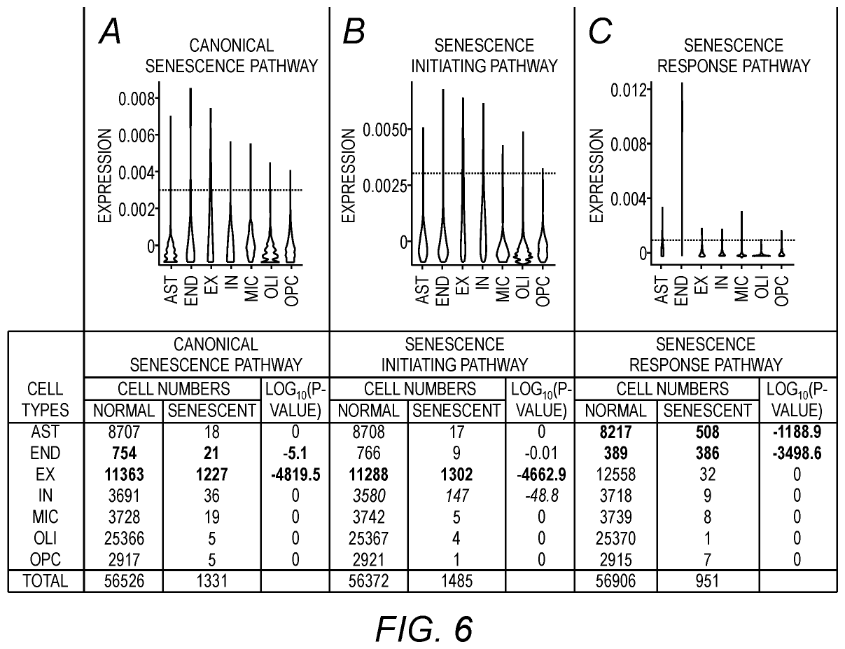

To determine which brain cell type(s) were represented in the senescent cell population, a hypergeometric test was used to categorize cells, as defined in the original studies^{22,23}. In cohort 1 excitatory neurons were the only cell population with senescence signatures more than expected across all three senescence gene sets (FIG. 1d-g). Subpopulations of astrocytes, endothelial cells and pericytes expressed gene patterns consistent with a senescence response (SRP), but not with canonical senescence to suggest an inflammatory phenotype not associated with senescence. Similarly, in cohort 2, the number of cells expressing CSP and CIP eigengenes were overrepresented in excitatory neurons. Astrocytes and endothelial cells were identified to express the SRP eigengene, which may reflect an inflammatory phenotype independent of a canonical senescence stress response^{28,29} (FIG. 6). Endothelial cells were also identified in the CSP eigengene, but only in cohort 2. These data indicate vascular cell senescence in the brain, which is well-established in cardiovascular disease¹⁴ and recently reported in human AD²⁸. Nonetheless, the predominant senescent cell population in both cohorts was excitatory neurons representing 97% and 92% of CSP cells in cohort 1 and cohort 2, respectively.

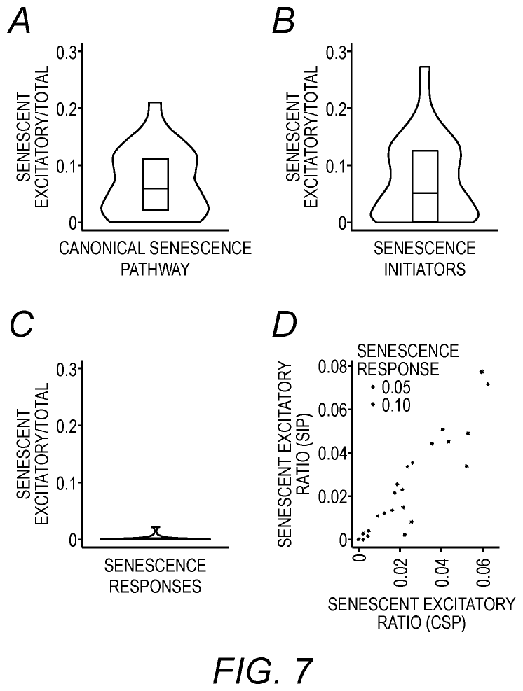

The relative proportion of excitatory neurons to total neurons within individual brains was determined next. As seen with total senescent cells, the proportion varied among individuals and ranged from 0-20%. On average, 4.2%, 3.5% and 2.5% cohort 1 excitatory neurons were also senescent as determined using the eigengenes for CSP, SIP and SRP, respectively (−log10 p-value: 3143, 2027, and 380, respectively), (FIG. 1h-j). In cohort 2, it was observed that more variability (0-30%) and a higher proportion of excitatory neurons expressing CSP and SIP (9.7% and 10.3%, respectively) with <1% expressing SRP (−log10 p-value: 4820, 4663, and 0, respectively; FIGS. 6 and 7). The three eigengenes (i.e., CSP, SIP and SRP) were designed to capture distinct aspects of cellular senescence that were reasoned would be associated in senescent cells, but not necessarily associated in non-senescent cells. Therefore, the correlation among the three senescence eigengenes within excitatory neurons was evaluated. The ratio of senescent excitatory neurons, as defined by CSP, highly correlated with SIP (Pearson correlation: 0.96) and SRP (0.90). Moreover, brains with higher proportions of senescent neurons displayed higher eigengene expression levels (FIG. 1k). Pathways were similarly correlated in cohort 2 (CSP and SIP: 0.94, FIG. 7). In summary, excitatory neurons represented the predominant senescent cell type across the 140,000 cells analyzed in these 76 brains as determined by three independent eigengenes (i.e., signatures) of cellular senescence.

### Senescent Excitatory Neurons With AD Neuropathology

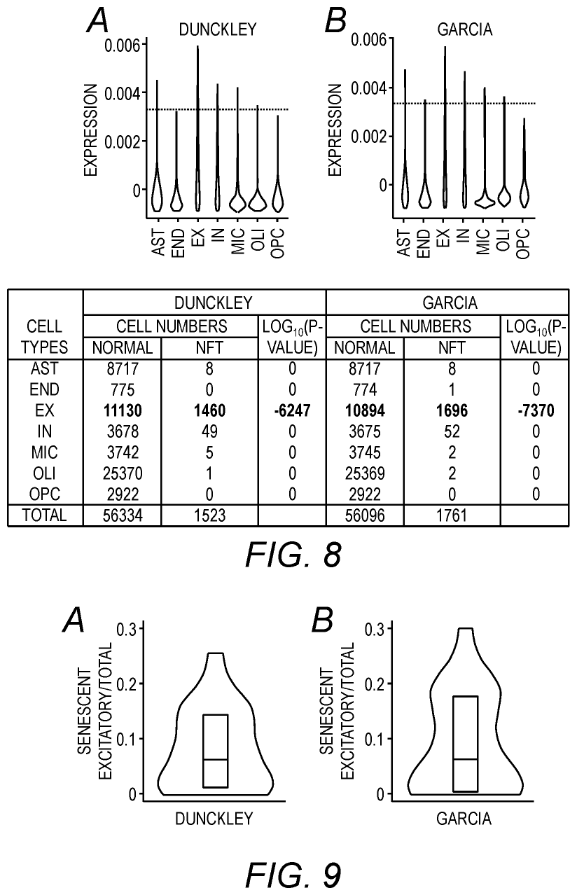

The accumulation of intraneuronal tau protein is the most common pathology across neurodegenerative diseases, including AD and chronic traumatic encephalopathy due to repeated head injury³⁰. Tau containing neurofibrillary tangles (NFTs) are characterized histologically by the presence of aggregated, phosphorylated misfolded tau proteins. They accumulate preferentially in excitatory neurons in human AD³¹ and drive cellular senescence in transgenic mice^{7,12}. Therefore, it was hypothesized that excitatory neurons containing NFTs may be senescent. To quantify the association between NFTs and senescence, eigengenes were created from two independent gene lists derived from laser capture microdissected neurons with NFTs^{32,33}, referred to as “NFT^{Duckley}” and “NFT^{Garcia}”, respectively. Using the NFT eigengenes, 1050 NFT^{Dunckley} cells and 1023 NFT^{Garcia} cells in cohort 1 reflecting 1.5% and 1.4% of the total cellular population, respectively (FIG. 2) were identified. Cohort 2 had slightly higher levels of NFT-bearing neurons, 1523 (2.6%) in cohort 2 and in cohort 1 and 1761 (3%) in cohort 2 (FIG. 8). The predominant cell type expressing the NFT eigengenes were excitatory neurons in both cohorts and with both eigengenes. These data are consistent with NFTs driving neuronal senescence in transgenic mice^{7,12} and preferentially accumulating in excitatory neurons in AD³¹.

As observed with senescent cells, the relative proportion of NFT-containing neurons to all excitatory neurons varied across individuals in cohort 1 (FIG. 2d-e) and cohort 2 (FIG. 9). Since the cell type (i.e., excitatory neurons) and proportions were consistent with other reports^{31,34}, it was interpreted that the eigengene could be used as a transcriptional surrogate to identify NFT-bearing neurons. Plotting NFT eigengene expression against senescence eigengene expression revealed a significant relationship between them (p=2e-16, adjusted R²=0.6803, m=0.863, FIG. 2f). To assess the association between senescent cells and NFT-bearing cells, hypergeometric tests was used. In cohort 1, 1485 CSP-senescent excitatory neurons and 1032 NFT^{Dunckley}-bearing excitatory neurons were identified. The expectation was to identify 44 overlapping cells if senescence and NFTs were not associated (i.e., co-expression by chance); however, 598 cells co-expressed both eigengenes indicating a significant association (−log10 adjusted p=1337). Similar results were obtained in cohort 2 and using the NFT^{Garcia} eigengene. These data confirmed the significant association whereby the senescence and NFT eigengenes were upregulated within the same cells.

Senescent and NFT neurons constituted a minor proportion of all neurons (i.e., of the 44, 172 total neurons analyzed in cohort 1, only 3.4% or 2.3% excitatory neurons met the criteria of senescence or NFTs, respectively). To visualize overlap between senescent and NFT neuron populations their distributions within the entire neuronal population were plotted. As shown in FIG. 3, the NFT-bearing neurons were found in the right-shifted neuronal cell population (i.e., overlapped with neurons expressing higher levels of the senescence eigengene). In other words, NFT-bearing neurons were the neurons that expressed the senescent eigengene greater than the mean. For example, the density plots indicated that <1% expressed the CSP eigengene lower than the mean (i.e., <1% of neurons with NFTs could be considered not senescent). However, caution was used not to label the remaining 99% of NFT-bearing neurons as senescent. The stringent cutoff required expression levels >mean+3sd. With these criteria, 35% of NFT-bearing neurons were identified as senescent and <1% as not senescent. The remaining 64% of NFT-bearing neurons could not be considered either senescent or not-senescent, but instead with upregulated senescent eigengene expression. These data revealed a continuum of senescence gene expression whereby 99% of NFT-bearing neurons displayed upregulated senescence eigengene greater than that of neurons without NFTs. Similar patterns were observed across eigengenes to confirm and validate the interpretation (FIG. 3a-c).

The distribution of the senescent neurons identified to contain NFTs was also determined (i.e., used the same experimental approach, but asked the question in the opposite direction). In cohort 1 all senescent cells expressed the NFT eigengene greater than the mean of all cells (FIG. 3d-f). Approximately 14% of the senescent neurons co-expressed the NFT eigengene >mean+3sd. The remaining 86% of cells had upregulated the NFT eigengene (4%, 34% and 48% mean+1sd, 2sd, 3sd, respectively), but did not reach >mean+3sd criteria. The data indicate significant overlap in cells co-expressing the senescence and NFT eigengenes; all neurons that expressed the NFT eigengene co-expressed the senescence eigengene greater than those without NFTs. Moreover, 99% of neurons that expressed the senescence eigengene co-expressed the NFT eigengene greater than those that were not senescence. Thus, there is overlap between senescent and NFT-bearing neurons.

### Example 2: CDKN2D/p19 Identified as a Molecular Regulator of NFT-Associated Neuronal Senescence

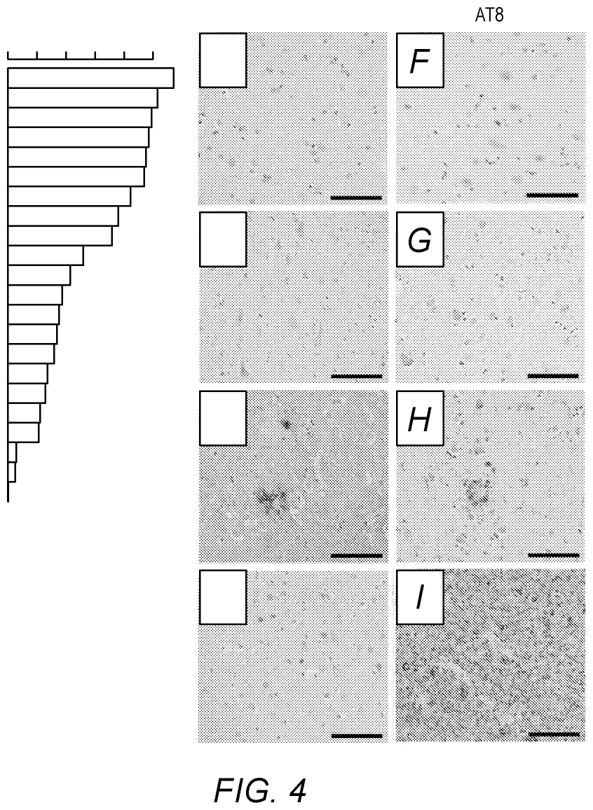

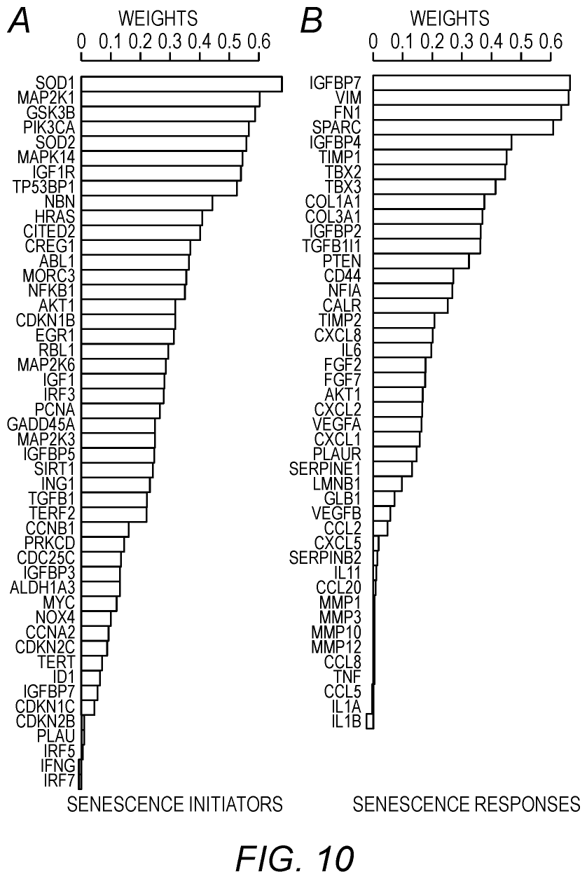

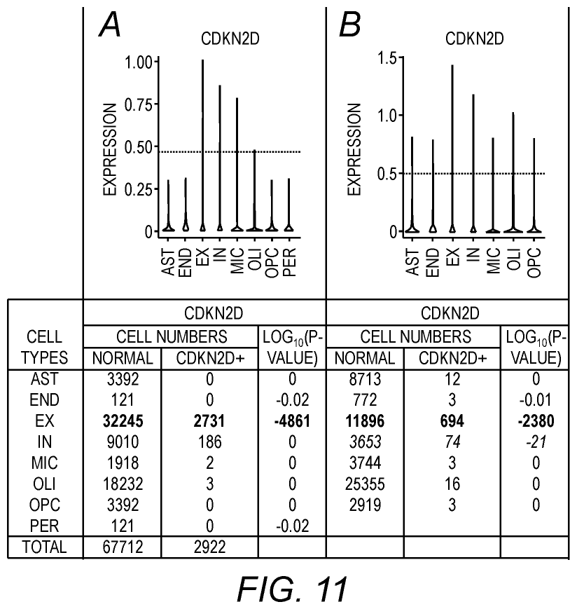

To gain insight into the mechanistic regulators of the senescence phenotype, the weight that each gene contributed to their respective senescence eigengenes was determined. Cyclin dependent kinase inhibitor 2D, CDKN2D, contributed most to the CSP eigengene (FIG. 4a; SIP and CRP: FIG. 10). Profiling single cells based on elevated CDKN2D expression resulted in a similar cellular composition as with the senescence eigengenes (i.e., predominantly excitatory neurons, FIG. 11). However, the number of senescent excitatory neurons was overestimated (CDKN2D: 2731 versus CSP eigengene: 1485) to indicate that not all cells with elevated CDKN2D could be considered senescent. To gain further insight into CDKN2D upregulation and association with senescent NFT-bearing neurons, immunohistochemistry was performed. Human cortex was analyzed from subjects with varying levels of AD tau neuropathology. Immunohistochemistry confirmed expression of the CDKN2D protein product, p19, in cases with AD pathology. Moreover, the staining revealed unique p19 subcellular localizations (FIG. 4b-e). Specifically, cytoplasmic p19 expression was present in early Braak stages as well as intermediate and advanced Braak stages, but more common in higher Braak stages. Punctate neuropil staining was also more common in advanced Braak stages. Intranuclear p19 immunostaining was observed in two cases which had a history of head injury. In addition, p19 immunostaining was observed in neuritic plaques, possibly highlighting dystrophic neurites, a feature of senescent mouse neurons⁸. Thus subcellular p19 localization may be a better informant regarding the senescence/NFT status of a neuron than total CDKN2D/p19 expression levels.

Using three separate human brain cohorts, three senescence transcriptomic profiles, two neuropathology profiles and histological methods, it was concluded that senescent cells in human brain are excitatory neurons with upregulated CDKN2D/p19 and NFT neuropathology. Additionally, the study generated necessary cellular and molecular data to guide future studies on the cellular senescence stress response in human brain. Results from the eigengene analyses indicated a continuum of senescence upregulation across cells which emphasizes the utility and importance of applying multi-analyte approaches when studying complex cellular stress responses. It provides a ranking of candidate genes and cellular profiles to be investigated in future studies.

### Bioinformatics/Biostatistics

Single nucleotide (sn) RNA-Seq data was downloaded from Mathys et. al, 2019²² and Zhou et. al, 2020²³ studies, which were available at Accelerating Medicines Partnership-Alzheimer's Disease (AMP-AD²⁴) website, using the synapser (r-docs.synapse.org/articles/synapser.html) R package³⁵ (Version 0.6.61). For each of the three gene sets in Table 2^{7,32}, the compute.pigengene( ) function from the Pigengene package (Version 1.13.4) was used to compute an eigengene²⁶, which is a weighted average expression over all genes in the corresponding list²⁷. Weights were optimized using a principal component analysis in a way that the explained variance was maximized, and thus, the loss of biological information is expected to be minimal. The mean expression of each eigengene was computed over all analyzed cells. A cell was considered to be senescent if the level of expression of the eigengene is more than the mean expression over all cells plus three times the standard deviation. A hypergeometric test was used to identify the cell types in which senescent cells are overrepresented. The project.eigen( ) function from the Pigengene package was used to infer the eigengenes values in the sn-RNA-Seq validation dataset based on the same weights that had been obtained from the analysis on the Mathys train dataset. In order to see how much senescence and NFT expressing eigengenes overlap, their expression was visualized by density plots using ggplot2 package in R. A kernel density estimate was used to represent the probability density function of eigengene values (FIG. 3). Moreover, the significance overlap of NFT cells that are also senescent is tested using hypergeometric test. The log10 of any p-value between 0.1 and 1 was replaced with ˜0.

### Immunohistochemical (IHC) Staining for p19

IHC stains were performed using a Thermo Scientific™ Lab Vision™ Autostainer 480 following deparaffinization of formalin-fixed paraffin-embedded sections (FFPE) and 30 minutes of heat-induced antigen retrieval in citrate buffer. Endogenous peroxidase was blocked by immersion in 3% hydrogen peroxide for 10′ and rinsed. A protein block for 15′ with 2.5% normal goat serum (Sigma) was then performed. After rinsing, sections were incubated with the rabbit polyclonal anti-p19 antibody (abcam) at 1:100 for 45′, washed and incubated with secondary antibody (goat anti-rabbit IgG (HRP), VisUCyte) for 45′ followed by rinsing. Diaminobenzidine (DAB) chromagen (BD Pharmigen) was used to visualize the immunoreactivity. IHC staining for p19 was performed on FFPE sections of the middle frontal gyrus from 6 Alzheimer disease (AD) cases, all of which demonstrated a high level of Alzheimer disease neuropathologic change (ADNC) with a Braak stage of VI, 3 intermediate ADNC level cases and 2 primary age-related tauopathy (PART) control cases (Braak stages I-II) with no neocortical neurofibrillary tangles.

See also Dehkordi et al., Nat Aging, December 2021 1(12): 1107-1116, which is incorporated by reference herein in its entirety.

TABLE 2
Figures Map

| | | Total Cell | Prominent | Neuron | |
|---|---|---|---|---|---|
| | Marker set | Ratios | Cell types | Ratios | Weights |
| Cohort | Senescence | FIG. 1 | FIG. 1 | FIG. 1 | FIG. 3, |
| 1 | pathways | | | | FIG. 10 |
| | Dunckley | NA | FIG. 2 | FIG. 2 | |
| | Garcia | NA | FIG. 2 | FIG. 2 | |
| | CDKN2D | NA | FIG. 11 | NA | NA |
| Cohort | Senescence | FIG. 5 | FIG. 6 | FIG. 7 | NA |
| 2 | pathways | | | | |
| | Dunckley | NA | FIG. 8 | FIG. 9 | NA |
| | Garcia | NA | FIG. 8 | FIG. 9 | NA |
| | CDKN2D | NA | FIG. 11 | NA | NA |

### REFERENCES

1 Rodier, F. & Campisi, J. Four faces of cellular senescence. J Cell Biol 192, 547-556, doi:10.1083/jcb.201009094 (2011).
2 Tchkonia, T., Zhu, Y., van Deursen, J., Campisi, J. & Kirkland, J. L. Cellular senescence and the senescent secretory phenotype: therapeutic opportunities. J Clin Invest 123, 966-972, doi:10.1172/JCI64098 (2013).
3 Hernandez-Segura, A., Nehme, J. & Demaria, M. Hallmarks of Cellular Senescence. Trends Cell Biol 28, 436-453, doi:10.1016/j.tcb.2018.02.001 (2018).
4 Coppe, J. P. et al. Senescence-associated secretory phenotypes reveal cell-nonautonomous functions of oncogenic RAS and the p53 tumor suppressor. PLoS Biol 6, 2853-2868, doi:10.1371/journal.pbio.0060301 (2008).
5 Kuilman, T. & Peeper, D. S. Senescence-messaging secretome: SMS-ing cellular stress. Nat Rev Cancer 9, 81-94, doi:10.1038/nrc2560 (2009).
6 Acosta, J. C. et al. A complex secretory program orchestrated by the inflammasome controls paracrine senescence. Nat Cell Biol 15, 978-990, doi:10.1038/ncb2784 (2013).
7 Musi, N. et al. Tau protein aggregation is associated with cellular senescence in the brain. Aging Cell 17, e12840, doi:10.1111/acel.12840 (2018).
8 Chow, H. M. et al. Age-related hyperinsulinemia leads to insulin resistance in neurons and cell-cycle-induced senescence. Nat Neurosci 22, 1806-1819, doi:10.1038/s41593-019-0505-1 (2019).
9 Ogrodnik, M. et al. Obesity-Induced Cellular Senescence Drives Anxiety and Impairs Neurogenesis. Cell Metab 29, 1061-1077 e1068, doi:10.1016/j.cmet.2018.12.008 (2019).
10 Riessland, M. et al. Loss of SATB1 Induces p21-Dependent Cellular Senescence in Post-mitotic Dopaminergic Neurons. Cell Stem Cell 25, 514-530 e518, doi:10.1016/j.stem.2019.08.013 (2019).
11 Chinta, S. J. et al. Cellular Senescence Is Induced by the Environmental Neurotoxin Paraquat and Contributes to Neuropathology Linked to Parkinson's Disease. Cell Rep 22, 930-940, doi:10.1016/j.celrep.2017.12.092 (2018).
12 Bussian, T. J. et al. Clearance of senescent glial cells prevents tau-dependent pathology and cognitive decline. Nature 562, 578-582, doi:10.1038/s41586-018-0543-y (2018).
13 Zhang, P. et al. Senolytic therapy alleviates Abeta-associated oligodendrocyte progenitor cell senescence and cognitive deficits in an Alzheimer's disease model. Nat Neurosci 22, 719-728, doi:10.1038/s41593-019-0372-9 (2019).
14 Tchkonia, T. & Kirkland, J. L. Aging, Cell Senescence, and Chronic Disease: Emerging Therapeutic Strategies. JAMA 320, 1319-1320, doi:10.1001/jama.2018.12440 (2018).
15 Saez-Atienzar, S. & Masliah, E. Cellular senescence and Alzheimer disease: the egg and the chicken scenario. Nat Rev Neurosci, doi:10.1038/s41583-020-0325-z (2020).
16 Justice, J. N. et al. Cellular Senescence Biomarker p16INK4a+ Cell Burden in Thigh Adipose is Associated With Poor Physical Function in Older Women. J Gerontol A Biol Sci Med Sci 73, 939-945, doi:10.1093/gerona/glx134 (2018).
17 Hickson, L. J. et al. Senolytics decrease senescent cells in humans: Preliminary report from a clinical trial of Dasatinib plus Quercetin in individuals with diabetic kidney disease. EBioMedicine 47, 446-456, doi:10.1016/j.ebiom.2019.08.069 (2019).
18 Katsouri, L. et al. Ablation of reactive astrocytes exacerbates disease pathology in a model of Alzheimer's disease. Glia 68, 1017-1030, doi:10.1002/glia.23759 (2020).
19 Hansen, D. V., Hanson, J. E. & Sheng, M. Microglia in Alzheimer's disease. J Cell Biol 217, 459-472, doi:10.1083/jcb.201709069 (2018).
20 Ogrodnik, M., Salmonowicz, H., Jurk, D. & Passos, J. F. Expansion and Cell-Cycle Arrest: Common Denominators of Cellular Senescence. Trends Biochem Sci 44, 996-1008, doi:10.1016/j.tibs.2019.06.011 (2019).
21 Sharpless, N. E. & Sherr, C. J. Forging a signature of in vivo senescence. Nat Rev Cancer 15, 397-408, doi:10.1038/nrc3960 (2015).
22 Mathys, H. et al. Single-cell transcriptomic analysis of Alzheimer's disease. Nature 570, 332-337, doi:10.1038/s41586-019-1195-2 (2019).
23 Zhou, Y. et al. Human and mouse single-nucleus transcriptomics reveal TREM2-dependent and TREM2-independent cellular responses in Alzheimer's disease. Nat Med 26, 131-142, doi:10.1038/s41591-019-0695-9 (2020).
24 Hodes, R. J. & Buckholtz, N. Accelerating Medicines Partnership: Alzheimer's Disease (AMP-AD) Knowledge Portal Aids Alzheimer's Drug Discovery through Open Data Sharing. Expert Opin Ther Targets 20, 389-391, doi:10.1517/14728222.2016.1135132 (2016).
25 Bennett, D. A. et al. Religious Orders Study and Rush Memory and Aging Project. J Alzheimers Dis 64, S161-S189, doi:10.3233/JAD-179939 (2018).
26 Foroushani, A. et al. Large-scale gene network analysis reveals the significance of extracellular matrix pathway and homeobox genes in acute myeloid leukemia: an introduction to the Pigengene package and its applications. BMC Med Genomics 10, 16, doi:10.1186/s12920-017-0253-6 (2017).
27 Oldham, M. C., Horvath, S. & Geschwind, D. H. Conservation and evolution of gene coexpression networks in human and chimpanzee brains. Proc Natl Acad Sci USA 103, 17973-17978, doi:10.1073/pnas.0605938103 (2006).
28 Bryant, A. G. et al. Cerebrovascular Senescence Is Associated With Tau Pathology in Alzheimer's Disease. Front Neurol 11, 575953, doi:10.3389/fneur.2020.575953 (2020).
29 Bhat, R. et al. Astrocyte senescence as a component of Alzheimer's disease. PLoS One 7, e45069, doi:10.1371/journal.pone.0045069 (2012).
30 Orr, M. E., Sullivan, A. C. & Frost, B. A Brief Overview of Tauopathy: Causes, Consequences, and Therapeutic Strategies. Trends Pharmacol Sci 38, 637-648, doi:10.1016/j.tips.2017.03.011 (2017).
31 Fu, H. et al. A tau homeostasis signature is linked with the cellular and regional vulnerability of excitatory neurons to tau pathology. Nat Neurosci 22, 47-56, doi:10.1038/s41593-018-0298-7 (2019).
32 Dunckley, T. et al. Gene expression correlates of neurofibrillary tangles in Alzheimer's disease. Neurobiol Aging 27, 1359-1371, doi:10.1016/j.neurobiolaging.2005.08.013 (2006).
33 Otero-Garcia, T. et al. Single-soma transcriptomics of tangle-bearing neurons in Alzheimer's disease reveals the signatures of tau-associated synaptic dysfunction. bioRxiv, doi:https://doi.org/10.1101/2020.05.11.088591 (2020).
34 Furcila, D., Dominguez-Alvaro, M., DeFelipe, J. & Alonso-Nanclares, L. Subregional Density of Neurons, Neurofibrillary Tangles and Amyloid Plaques in the Hippocampus of Patients With Alzheimer's Disease. Front Neuroanat 13, 99, doi:10.3389/fnana.2019.00099 (2019).
35 Team, R. C. R: A language and environment for statistical computing, <https://www.R-project.org/> (2013).
36 Otero-Garcia, M. et al. Single-soma transcriptomics of tangle-bearing neurons in Alzheimer's disease reveals the signatures of tau-associated synaptic dysfunction (bioRxiv, 2020).
37 Chan, J. H., Lim, S., and Wong, W. S. (2006). Antisense oligonucleotides: from design to therapeutic application. Clin. Exp. Pharmacol. Physiol. 33, 533-540. doi: 10.1111/j.1440-1681.2006.04403.
38 Bennett, C. F., Baker, B. F., Pham, N., Swayze, E., and Geary, R. S. (2017). Pharmacology of antisense drugs. Annu. Rev. Pharmacol. Toxicol. 57, 81-105. doi: 10.1146/annurev-pharmtox-010716-104846
39 Crooke, S. T. (2017). Molecular mechanisms of antisense oligonucleotides. Nucleic Acid Ther. 27, 70-77. doi: 10.1089/nat.2016.0656
40 Di Fusco D, Dinallo V, Marafini I, Figliuzzi MM, Romano B and Monteleone G (2019) Antisense Oligonucleotide: Basic Concepts and Therapeutic Application in Inflammatory Bowel Disease. Front. Pharmacol. 10:305. doi: 10.3389/fphar.2019.00305

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A method of detecting neurofibrillary tangles in a subject comprising:
a. assaying for the expression of CDKN2D in the brain of the subject; and
b. comparing the amount of CDKN2D expression to a control, whereby increased CDKN2D expression relative to the control is indicative of the presence of neurofibrillary tangles in the subject.

2. The method of claim 1, wherein the method is in vitro and the method comprises obtaining a sample from the subject.

3. The method of claim 2, wherein the sample is cerebral spinal fluid or brain tissue.

4. The method of claim 1, wherein the method is in vivo and the detecting comprises imaging (e.g. PET imaging).

5. The method of claim 1, wherein the method comprises administering a detectable compound (e.g. polynucleotide or antibody) specific for a CDKN2D expressed polynucleotide (e.g., mRNA) or protein (p19INK4D), and further comprising detecting the compound.

6. A method of treating a disease associated with the presence of neurofibrillary tangles, comprising administering a treatment for the disease to a subject identified as having neurofibrillary tangles by a method of claim 1.

7. The method of claim 6, wherein the disease associated with neurofibrillary tangles is an age-related disease.

8. The method of claim 6, wherein the disease associated with neurofibrillary tangles is a tauopathy.

9. The method of claim 6, wherein the disease is selected from mild cognitive impairment, Alzheimer's disease, traumatic brain injury, primary age-related tauopathy (PART), neurofibrillary tangle-predominant dementia (NFTPD), Pick disease, Parkinson's disease, Chronic traumatic encephalopathy (CTE), progressive supranuclear palsy (PSP), frontotemporal dementia, frontotemporal lobar degeneration, progressive supranuclear palsy, corticobasal degeneration, Amyotrophic Lateral Sclerosis (ALS), and Huntington's Disease.

10. The method of claim 6, wherein the administering is by direct administration to the brain of the subject.

11. The method of claim 6, wherein the treating comprises inhibiting the formation of, or reducing the presence of, neurofibrillary tangles in the subject.

12. The method of claim 6, wherein the treating inhibits the expression or activity of CDKN2D or p19INK4D.

13. The method of claim 6, wherein the treating inhibits cellular senescence caused by or associated with neurofibrillary tangles in a subject.

14. The method of claim 6, wherein the treatment comprises a genetic modifying agent, antibody or fragment thereof.

15. The method of claim 14, wherein the genetic modifying agent comprises an antisense oligonucleotide, an RNAi, an siRNA, or a gene editing system selected from a CRISPR system, a zinc finger nuclease system, and a TALE system.

16. The method of claim 14, wherein the treatment comprises a therapeutic antibody or fragment thereof that specifically binds to the protein encoded by CDKN2D.

17. The method of claim 14, wherein the genetic modifying agent, antibody or fragment thereof comprises a detectible group.

18. The method of claim 17, further comprising performing PET imaging on the subject.

19. A method of monitoring the progress of a neurofibrillary-associated disease in a subject comprising:
a. detecting a first level of CDKN2D expression in a biological sample obtained from the subject at a first time point;
b. detecting a second level of CDKN2D expression in a biological sample obtained from the subject at a second time point; and
c. comparing the second level of CDKN2D expression with the first level of CDKN2D expression,
wherein said comparison indicates the progress of the neurofibrillary-associated disease in the subject.

20. The method of claim 19, wherein the first time point is a time point before initiation of a treatment regimen, and wherein the second time point is a time point after initiation of a treatment regimen.
